(19) United States
(12) Patent Application Publication
(10) Pub. No.: US 2020/0371117 A1
(43) Pub. Date: Nov. 26, 2020

(54) DETECTION OF CREATINE LEVELS USING ENZYME COMPOSITIONS

(71) Applicant: IP2IPO Innovations Limited, London (GB)

(72) Inventors: Robert M. Learney, London (GB); Martyn G. Boutelle, London (GB)

(21) Appl. No.: 16/636,598

(22) PCT Filed: Aug. 3, 2018
(86) PCT No.: PCT/GB2018/052231

§ 371 Date: Feb. 4, 2020

(30) Foreign Application Priority Data
Aug. 4, 2017 (GB) 1712592.3

(51) Int. Cl. G01N 33/70; C12Q 1/26; C12Q 1/34; G01N 33/574

(52) U.S. Cl. CPC G01N 33/70; C12Q 1/26; G01N 2333/98; G01N 33/57438; G01N 2333/902; C12Q 1/34

(57) ABSTRACT

The invention provides compositions and systems that allow the sensitive determination of the level of creatinine in a particular solution. Through the optimisation of enzymatic methods to detect creatinine the real-time determination of creatinine levels and creatinine clearance rates are also provided, allowing the real-time monitoring of kidney function. This is considered to be useful both in the monitoring of live subjects, and in the monitoring of isolated organs, such as a kidney, intended for transplantation.

### FIELD OF THE INVENTION

The invention provides compositions, and systems that allow the sensitive determination of the level of creatinine in a particular solution. Methods of using the compositions and systems in the real-time determination of creatinine levels and creatinine clearance rates are also provided, allowing the real-time monitoring of kidney function.

### BACKGROUND

Although the kidney has many different components, such as the nephron and the glomerulus, the function of which can individually be impaired, current methods to determine the kidney function of a subject assess the overall performance of the kidney and at present it is not possible to determine which precise part of the kidney is affected. This overall measure of kidney function is called the glomerular filtration rate (GFR) and assesses the ability of the kidney to clear substances, specifically creatinine, from the blood. This is the method routinely used in the clinical setting.

The GFR is often reported as a value in ml/minute normalised to a body surface area of 1.73 m². The normal adult GFR lies between 90 ml/min/1.73 m² and 130 ml/min/1.73 m², worsening GFR is the clinical means of assessing the stage of a patient's chronic kidney disease, where a GFR of 15 ml/min or less then is termed end stage renal failure.

The equation used to calculate GFR, based on the clearance of creatinine is:

$$G F R ( ml \text{/} min ) = Urine Flow Rate \cdot ( \frac{[ Urine ]}{[ Plasma ]} )$$

Small amounts of creatinine are also secreted from the distal tubules, though the amount secreted remains constant even in the face of declining renal function.

Creatinine is present in human blood in micromolar concentrations, because of the constant filtration by the kidney. In a steady state system, the body's skeletal muscles will release a constant amount of creatinine into the bloodstream, and the kidneys will remove this from the circulation through a combination of filtration and active tubular secretion.

This active tubular secretion comprises a larger fraction of creatinine clearance at the lower functional extreme, and leads to overestimation of the glomerular filtration rate (GFR).

Measuring Creatinine in Clinical Practice

Three main techniques are presently used for measuring creatinine concentrations in clinical samples: (i) the Jaffe reaction, (ii) enzymatic methods, and (iii) isotope dilution mass spectrometry (IDMS), which is the method against which all other methods are now compared.

IDMS

IDMS is considered to be the most accurate method of quantifying analytes of interest in modern clinical biochemistry. The principle is simple, and akin to estimating wild populations of animals by tag-and-release methods. Beginning with a sample of unknown quantity yet known isotopic composition and diluting it with a standard of known quantity and isotopic composition, one is able to determine the concentration in the original sample by measuring the final dilution ratio of the isotope in question. This method combines internal ratiometric normalisation with the high precision and low limits of detection of modern mass spectroscopy, leading to highly accurate and reproducible results with low bias.

Unfortunately, the methodology and the size and cost of GC-MS devices required to undertake this assay do not lend themselves to miniaturisation for incorporation into a continuous in-line creatinine assay.

The Jaffe Reaction

This method of detecting creatinine predates its recognition as an important indicator of renal function. The first formal description of the production of a coloured compound following the alkalinisation of the reaction product of picric acid and urinary creatinine was published by Max Jaffe (1841-1911) in 1886, for whom this detection method is named. The intensity of the colour change, and thus the amount of creatinine in the sample, could be rapidly assessed with colourimetry or spectrophotometry with maximum absorbance at 520 nm.

Unfortunately, the method is not without its drawbacks, not least of which is the historic lack of standardisation between laboratories, demonstrated by the work leading to the development of the IDMS standards [35]. The Jaffe reaction is also highly non-specific, and can produce false positives or negatives with a vast number of endogenous and exogenous compounds often found in human samples, including trace amounts of protein, glucose, ketone bodies, bilirubin and certain aminoglycoside and cefalosporin antibiotics.

Attempting to calibrate against these can in fact introduce greater uncertainty to values on the borderline between normal and abnormal function, and paradoxically underestimate the creatinine concentration of urine where none of the endogenous interferents are found [37]. As an illustration of the impact of even small errors, an increase of just 20 μmol in the absolute value of the serum creatinine concentration can mean the difference between normal function and early renal failure.

For these reasons, the Jaffe method is being slowly replaced by enzymatic detection in the developed world.

Three-Enzyme System

This method relies on a more complicated three-step digestion of creatinine into hydrogen peroxide, formaldehyde and glycine in a 1:1:1 molar ratio, via creatine and sarcosine as intermediates, and urea as an intermediate by-product.

This system results in two potential targets for non-optical detection—urea and H₂O₂.

Detecting Urea

The detection of urea requires a further coupling reaction to urease which catalyses the production of NH₃ and CO₂. Whilst the detection of NH₃ is complicated by difficulties, CO₂ production can be quantified with a common Severinghaus electrode, or more exotic doped nanomaterials [51].

The Severinghaus electrode requires a particular internal configuration of a glass pH electrode encased within a solution of NaHCO₃ of known pH, and separated from the sample solution by a gas-permeable membrane. As CO₂ passes through the membrane, it dissolves into the NaHCO₃ solution to evolve H⁺ ions. These are then potentiometrically sensed at the internal pH electrode.

Whilst the principle is well understood, and the sensor behaves in a linear manner over the normal range of human blood pCO₂, this triple-walled, liquid-containing sensor is very hard to miniaturise, and only a handful of reports exists in the literature of micromachined Severinghaus-type electrodes, with very slow response times [52].

Furthermore, the amount of CO₂ evolved from the complete digestion of creatinine will be in the sub-millimolar range at best. This means that any CO₂ sensor system will be exposed to an offset of tens of times the expected signal magnitude from the background levels of CO₂ dissolved in the sample from normal metabolism (4.5-6 kPa, 1.75≡2.33 mmol), whether that sample is drawn from the blood or the urine.

Finally, any biological sample will also contain levels of urea that are also far greater than that of creatinine.

Detecting H₂O₂

H₂O₂ occurs within the blood and urine as a result of oxidative metabolic processes, but at low micromolar levels which rapidly diminish under the effects of endogenous antioxidants in the plasma including catalase, haeme, and ascorbate [53]. Thus, the only appreciable source of H₂O₂ in the triple-enzyme scheme is the creatinine itself via sarcosine oxidase. H₂O₂ is also readily detectable through amperometry.

Finally, the overall equilibrium of the triple-enzyme system lies far to the right with the generation of products and consumption of the substrate, unlike that of detecting creatinine deiminase via glutamate dehydrogenase and glutamate oxidase. This indicates that a higher potential level of product, and thus signal, will result from a smaller quantity of substrate, improving the signal to noise ratio and limits of detection for this system.

Microdialysis

Microdialysis is a method for obtaining continuous samples of small molecules from a tissue or solution of interest, whilst minimising interferents, and was originally pioneered in the 1970s for sampling neurotransmitters from the rat brain [55]. It works by continuously perfusing one side of a semi-permeable membrane with a fluid which lacks the molecule(s) of interest so that target molecules will diffuse down their concentration gradients across the membrane into the perfusate. At the same time, molecules above the cut-off weight of the membrane, or which are already in equilibrium with the perfusate, will not change in concentration. The post-membrane dialysate then carries the target molecule to the detection system.

Microfluidics

The term ‘microfluidics’ describes the practice of working with volumes of liquid at or below the nanolitre scale, with flow channels only tens to hundreds of microns in diameter. Unlike traditional laboratory analyses, operating on these scales brings powerful advantages in terms of reducing the required volumes of samples and potentially costly reagents, whilst improving sensitivity, reproducibility and the speed of analysis [56]. This is particularly useful for enzyme-based reactions, where the enzymes themselves may be particularly costly, and where only small amounts of substrate may be available, as is the case with microdialysis.

Labsmith Platform

The LabSmith Microfluidic Platform system (LabSmith, Inc., Livermore, Calif., USA) is compatible with 150 μm internal diameter inert PEEK (Poly Ether Ether Ketone) tubing (360 μm outer diameter), with customised substrate and reactant reservoirs on the millilitre scale, precision micropumps capable of handling microlitre volumes to create flow rates down to ≈8 nanolitres per second (500 nl/min), and three or four-way switching valves with internal PEEK surfaces. All of these components are fully modular and exchangeable with a common locking ferrule fitting for creating watertight microfluidic connections, and a screw-fit breadboard system for holding the various other components in place.

Amperimometric Sensors

Amperometry is the technique of measuring the number of electrons consumed or produced by a redox reaction at a certain electrical potential, such as that invented by Leyland Clark (1918-2005) in the 1950s for measuring the partial pressure of oxygen in solution at a potential of −0.6V to −0.7V vs. AgIAgCl.

An amperometric sensor comprises three elements—(i) a working electrode to carry out the redox reaction with the substrate of interest, (ii) an auxiliary or counter-electrode to balance the other side of the redox reaction, and (iii) a reference electrode to fix the circuit at a stable point in electrical space.

A potentiostat circuit uses a servo amplifier to automatically adjust the current flow from the counter-electrode to maintain the potential of the working electrode at a fixed point from the reference to control the redox reaction, and is combined with a transimpedance amplifier to measure the current passed by the working electrode as a voltage signal for recording and analysis.

The transimpedance amplifier must have a suitably high input impedance on the order of 10\hat{ }12Ω to prevent any interference with the redox reaction at the working electrode, and a frequency response to match the expected changes in the system's redox rate with the presentation of new substrate. Similarly, the servo amplifier must have a sufficiently low output impedance and response rate to be able to maintain the stability of potential at the working electrode.

The three-enzyme system has been used in the prior art to determine the level of creatinine.

Tsuchida and Yoda [40] use a three-enzyme system. The authors determined that the optimum pH of sarcosine oxidase in free solution is pH 7.5, but once immobilised it increases to pH 10. Therefore, one would expect that the optimal pH of the free solution three-enzyme system would be around pH 7.5.

Khue et al [72] uses electrodes comprising immobilised enzymes. The optimal pH of the system, out of a tested range of pH 6.5-8.5, was found to be pH 8.0. Subsequent experiments were performed at physiological pH in PBS buffer.

Sakslund et al [74] found that the optimal pH of the three-enzyme system, immobilised on to electrodes, was pH 7.7.

Madaras [77] discusses an electrode on which the enzymes of the three-enzyme system are immobilised in a layer. The detection limit of this system was 30 uM creatinine, performed at pH 7.3-7.4 in PBS.

There is a need for a portable, low-cost, system for continuously sampling and assaying normal creatinine concentrations in either the blood or urine of an isolated perfused kidney.

It was only through the work of the present invention that the optimal pH for a sensor system utilising the three-enzyme system with enzymes in free solution was determined.

### BRIEF SUMMARY OF THE INVENTION

The prior art methods of determining kidney function, as discussed above, are inadequate and out-dated. The present inventors have surprisingly found that the determination of creatinine levels using a three-enzyme system in free solution, rather than the prior art approaches of having at least one enzyme embedded on the electrode, gives surprisingly accurate and sensitive readouts, sufficiently so to allow the real-time determination of kidney function. Without wishing to be bound by any theory, the inventors consider that the fact that the enzyme solution allows the reaction to go to near completion, thus generating a signal which is higher than that obtained by the prior art biosensors where the enzymes are only exposed to the substrate for a short time, is at least partly responsible for this improvement.

In addition, and contrary to the teachings of the prior art, for example Tsuchida and Yoda [40] the inventors have found that the optimal pH of the three-enzyme system in free solution is actually at a relative high pH, i.e. pH 8.0-8.6. This optimisation is considered to further enhance the sensitivity of the determination.

The three-enzyme free solution approach in combination with detection of the resultant H₂O₂ by an amperometric sensor is considered to give particularly surprising sensitivity and allows the real-time detection of creatinine at medically relevant levels, for the first time.

### DETAILED DESCRIPTION OF THE INVENTION

A first aspect of the invention provides a composition comprising any two of or all of creatininase, creatinase and sarcosine oxidase.

In one embodiment the composition is a liquid. In another embodiment the composition is a solid. By a solid we mean for instance that the components of the composition are provided as a dry powder, rather than that the components are embedded in or on an electrode. In a further embodiment the composition is in the form of a gel.

In one embodiment the composition comprises creatininase and creatinase. In a further embodiment the composition comprises creatininase and sarcosine oxidase. In yet a further embodiment the composition comprises sarcosine oxidase and creatinase. In another embodiment the composition comprises creatininase, creatinase and sarcosine oxidase.

The three-enzyme system referred to here utilises all three of creatininase, creatinase and sarcosine oxidase. However, the skilled person will understand that for the three-enzyme system to be employed all three enzymes do not have to be in the same composition. For example a composition of the invention comprising creatininase and creatinase may be allowed to react with the substrate, followed by the subsequent addition of sarcosine oxidase to produce the hydrogen peroxide that can be detected by the sensor. References to the three-enzyme system herein may therefore refer to the use of all three enzymes simultaneously, i.e. in the same composition, or the sequential addition of the enzymes.

Although in some embodiments the two or more enzymes of the composition are cross-linked, for example by glutardialdehyde, either to each other or to another agent such as BSA, in a preferred embodiment the enzymes are not cross-linked.

Accordingly, in one embodiment the invention provides the composition of the invention wherein the enzymes are not cross-linked, optionally have not been cross-linked with glutardialdehyde.

In one embodiment the invention provides the composition wherein at least one, optionally two, optionally all of the enzymes are not immobilised, optionally wherein all of the enzymes are in solution.

It will be appreciated that one intended use of the composition is in the determination of the level of creatinine, or the steady state real-time monitoring of the level of creatinine. Accordingly, in one embodiment the composition is defined by the requirements of the actual reaction mix, i.e. when the composition of the invention is mixed with a sample comprising a substrate, for example creatinine, and in which hydrogen peroxide is generated. For instance, the composition may comprise the enzymes at a particular concentration, or in a particular buffer at a particular concentration or pH such that in the in the final mixed solution that results from the mixing of the sample, for example a dialysate, which contains the creatinine and the enzyme composition of the present invention various parameters are met.

For instance it is well known to supply a composition comprising concentrated amounts of various components such that upon dilution the required concentrations are arrived at. This is well known to the skilled person. Accordingly the composition of the invention can be produced to allow any preferred final reaction concentrations or parameters defined herein to be met. For instance in some embodiments the composition of the invention is used along with microdialysis and the enzyme mixture is mixed with the microdialysate at a particular flow rate. The skilled person will be able to determine, based on the flow rate and the parameters involved, a suitable starting composition of the invention that, when in use, provides the required parameters.

In one embodiment, where the composition of the invention is a liquid, the liquid comprises a buffer. Accordingly, one embodiment of the invention provides the enzymes of the composition of the invention in a buffer.

In another embodiment, where the composition of the invention is a gel, the gel may also comprise a buffer. Preferences for the buffer are as described herein.

The chosen buffer is considered to have a significant effect on the activity of the enzymes and the resultant sensitivity of the detection of creatinine. Prior art attempts to use the three-enzyme system have focussed on the use of the enzymes generally at a physiological pH and in phosphate buffered saline (PBS). Examples of these attempts are given FIG. 19. Most of this work also used biosensors wherein at least one of the enzymes of the three-enzyme system is incorporated into one of the electrodes. PBS was considered to be a suitable buffer for use with electrochemical sensors since a favourable interaction occurs between the phosphate and the electrodes.

However, despite the abundant use of PBS in the prior art, the inventors surprisingly found that PBS is not the most suitable buffer for use with the present invention. This may be because PBS has the ability to sequester divalent cations, such as Zn²⁺, Mn²⁺ and Mg²⁺, all of which are important cofactors for the creatininase enzyme isolated from various species. PBS is considered to form insoluble salts with cations such as these. FIG. 18 lists the solubility of various buffer salts, from which the skilled person can readily determine which are and which are not suitable buffers for use in the present invention. FIG. 18 illustrates the level of insolubility of phosphate salts of divalent cations, for example.

In a preferred embodiment, the buffer does not compete with creatininase for a cofactor of creatininase, for example a divalent cation cofactor, for example Zn²⁺, Mn²⁺ or Mg²⁺. In another embodiment the buffer does not sequester cations, for example divalent cations, for example Zn²⁺, Mn²⁺ or Mg²⁺.

Accordingly in one embodiment the buffer is not a phosphate buffer or PBS.

In one embodiment Tris based buffers are also not considered to be suitable for use with the composition of the invention. Accordingly, in one embodiment the buffer is not PBS and/or is not a Tris based buffer.

Since it is considered that the optimal pH for the reaction comprising all three enzymes (creatininase, creatinase and sarcosine oxidase) is between around pH 8.0 to pH 8.95, in one embodiment of the invention the buffer is a buffer that has a pKa of between 7.0 to 9.0. In one embodiment the buffer has a pKa of between 7.0 and 9.0 but is not PBS or tetraborate or Tris. In another embodiment the pKa of the buffer is between 7.05 and pH 8.95, optionally between 7.1 and 8.9, optionally between 7.15 and 8.85, optionally between 7.2 and 8.80, optionally between 7.20 and 8.75, optionally between 7.25 and 8.70, optionally between 7.30 and 8.65, optionally between 7.35 and 8.60, optionally between 7.40 and 8.55, optionally between 7.45 and 8.50, optionally between 7.40 and 8.45, optionally between 7.45 and 8.40, optionally between 7.50 and 8.35, optionally between 7.55 and 8.30, optionally between 7.60 and 8.25, optionally between 7.65 and 8.20, optionally between 7.70 and 8.15, optionally between 7.75 and 8.10, optionally between 7.80 and 8.05, optionally between 7.85 and 8.00, optionally between 7.90 and 7.95, or the pKa is at least any of the above mentioned pKa values, or is less than any of the above pKa values.

In one embodiment the pKa of the buffer is between 7.3 and 8.95.

In one embodiment the pKa of the buffer is 8.5 or around 8.5.

For any of the above pKa ranges, in one embodiment the buffer is not PBS and/or is not tetraborate and/or is not Tris and/or is not HEPES.

The skilled person will be well aware of, and has access to lists of, the pKa of various buffers.

In one embodiment, a particular example of a buffer that is considered to be useful with the present invention EPPS 4-(2-Hydroxyethyl)-1-piperazinepropanesulfonic acid. In another embodiment, HEPBS (N-(2-Hydroxyethyl)piperazine-N′-(4-butanesulfonic acid)) is also considered to be useful. In a further embodiment POPSO (Piperazine-1,4-bis(2-hydroxypropanesulfonic acid)), HEPPSO (N-(2-Hydroxyethyl) piperazine-N′-(2-hydroxypropane-3-sulfonic acid)) and MOBS (4-(N-Morpholino)butanesulfonic acid) are also considered to be useful.

It is considered that in one embodiment a buffer should be used within 1 pH unit of its pKa.

Buffers with a pKa of greater than 9 may also be used. However in the context of determining the creatinine levels of for instance blood or urine, a pKa of above 9.5 is unlikely to be useful. However, such a buffer, i.e. one with a pKa of greater than 9, or greater than 9.5 may be useful in other contexts and is also included as part of the invention.

Detailed information regarding the various properties of different buffers, including the pKa, can be readily located, for instance the Sigma website has detailed information on many buffers(http://www.sigmaaldrich.com/life-science/core-bioreagents/biological-buffers/learning-center/buffer-reference-center.html).

In one embodiment, the buffers of the present invention are used at room temperature, for example, at between 18° C. and 25° C., for example at 18° C., 19° C., 20° C., 21° C., 22° C., 23° C., 24° C. or 25° C. The buffer may typically be used at 20° C. In a further embodiment, the buffers of the present invention are used at temperatures above room temperature, for example, 26° C., 27° C., 28° C., 29° C., 30° C., 31° C., 32° C., 33° C., 34° C., 35° C. or higher. In a further embodiment, the buffer is used at a temperature of 55° C. or less, for example, 50° C., 45° C., 40° C. or lower. In a further embodiment, the buffer is not used at temperatures below room temperature.

In addition to the pKa of the buffer used, the pH of the reaction mix in which the enzymes are to perform is also very important. In one embodiment the pH of the composition or the buffer is between around pH 8.0 to pH 8.95. In one embodiment of the invention the composition or the buffer is a composition or the buffer that has a pH of between 7.0 to 9.0. In one embodiment the composition or the buffer has a pH of between 7.0 and 9.0 but is not PBS or tetraborate or Tris. In another embodiment the pH of the composition or the buffer is between 7.05 and pH 8.95, optionally between 7.1 and 8.9, optionally between 7.15 and 8.85, optionally between 7.2 and 8.80, optionally between 7.20 and 8.75, optionally between 7.25 and 8.70, optionally between 7.30 and 8.65, optionally between 7.35 and 8.60, optionally between 7.40 and 8.55, optionally between 7.45 and 8.50, optionally between 7.40 and 8.45, optionally between 7.45 and 8.40, optionally between 7.50 and 8.35, optionally between 7.55 and 8.30, optionally between 7.60 and 8.25, optionally between 7.65 and 8.20, optionally between 7.70 and 8.15, optionally between 7.75 and 8.10, optionally between 7.80 and 8.05, optionally between 7.85 and 8.00, optionally between 7.90 and 7.95, or the pH is at least any of the above mentioned pKa values, or is less than any of the above pH values.

In one embodiment the pH of the composition or the buffer is between 7.3 and 8.95.

In one embodiment the pH of the composition or the buffer is 8.5 or around 8.5.

For any of the above pH ranges, in one embodiment the buffer is not PBS and/or is not tetraborate and/or is not Tris and/or is not HEPES.

In one embodiment, the composition of the invention comprises a buffer at a concentration of between 5 mM and 100 mM, optionally between 10 mM and 90 mM, optionally between 15 mM and 85 mM, optionally between 20 mM and 80 mM, optionally between 25 mM and 75 mM, optionally between 30 mM and 70 mM, optionally between 35 mM and 65 mM, optionally between 40 mM and 60 mM, optionally between 45 mM and 55 mM, optionally 50 mM. The skilled person will readily be able to determine the appropriate concentration of buffer required. For instance, the skilled person may (i) Use the Henderson-hasselbalch equation to, for instance, calculate for normal human serum at pH 7.35 to get the lowest end of the appropriate range, keeping range within 0.1 of, for example, pH 8.5 and then (ii) for a basic pKa to buffer within 0.1 pH of 8.5, for example.

It will be appreciated that although the pH of the composition of the invention may be at a certain pH, upon addition of the biological sample, for instance blood, during or tissue fluid samples, the pH of the resultant mixture may vary. Preferably the variation in pH is kept to the minimum since in one embodiment the pH of the buffer of the composition is considered to be the optimal for the three-enzyme system and the time take to reach the T₉₀ may be extended if optimal conditions are not maintained. In one embodiment the pH of the resultant mixture is between 0-0.1 pH units different to the pH of the composition of the invention. In another embodiment the pH of the resultant mixture is between 0.1-0.2 pH units different to the pH of the composition of the invention. In another embodiment the pH of the resultant mixture is between 0.2-0.3 pH units different to the pH of the composition of the invention. In another embodiment the pH of the resultant mixture is between 0.3-0.4 pH units different to the pH of the composition of the invention. In another embodiment the pH of the resultant mixture is between 0.5-0.6 pH units different to the pH of the composition of the invention. In another embodiment the pH of the resultant mixture is between 0.6-0.7 pH units different to the pH of the composition of the invention. In another embodiment the pH of the resultant mixture is between 0.7-0.8 pH units different to the pH of the composition of the invention. In another embodiment the pH of the resultant mixture is between 0.8-0.9 pH units different to the pH of the composition of the invention. In another embodiment the pH of the resultant mixture is between 0.9-1.0 pH units different to the pH of the composition of the invention.

In another embodiment the pH of the buffer of the composition is not considered to be the optimal for the three-enzyme system but is designed such that once the composition of the invention has been mixed with the biological sample, for instance blood, during or tissue fluid samples, the optimal pH is obtained.

In one embodiment the composition comprises EPPS at pH 8.0-8.5, optionally 50 mM EPPS at pH 8.0-8.5, optionally 50 mM EPPS at pH 8.0 or 50 mM EPPS at pH 8.5.

In a preferred embodiment, the composition comprises 50 mM EPPS at pH 8.0 or pH 8.5.

As discussed above the various properties, including the pH, of different buffers are known to the skilled person, and the skilled person can readily determine which buffers are suitable for use with the present invention, based on the details given here in combination with the common general knowledge.

The combination of these buffer properties in conjunction with a solution of creatininase, creatinase and sarcosine oxidase was not previously contemplated in the prior art. It was only the work of the present inventors that identified the optimal pH of the three-enzyme reaction, and the unsuitability of PBS as the buffer.

Since it is the pH of the reaction mix that is important it will be appreciated that although the composition comprising the enzymes may also comprise a buffer as described herein, the buffer may instead be supplied separately, for instance as part of a kit of parts along with one or more or all of creatininase, creatinase and sarcosine oxidase. In this case one or more of the enzymes is added to the reaction mix separately to the buffer, which maintains the appropriate pH.

In one embodiment the only entities in the composition are creatininase, creatinase and sarcosine oxidase, and the buffer if the buffer is present. In this case, the composition of the invention consists of, or consists essentially of any two of or all of creatininase, creatinase and sarcosine oxidase, and the buffer as described above, where present. It will be appreciated that if the composition is a solid, then it is possible that the composition consists only of any two of or all of creatininase, creatinase and sarcosine oxidase. However, where the composition is a liquid of a gel, the composition must also comprise the liquid or gel component, which in one embodiment is not considered to have any material effect on the workings of the invention, and so the composition in this case consists or consists essentially of any two of or all of creatininase, creatinase and sarcosine oxidase.

However, it will be appreciated that in a situation such as monitoring kidney function, it may also be useful to, for example, monitor other metabolites or parameters of the subject. Accordingly, in some embodiments, the composition comprises the above agents in addition to possible also comprising other useful agents. For instance, it is considered to be useful if the composition also comprised urease, to allow the detection of urea, though the skilled person will appreciate that this reaction does not produce an electrochemical substance, and means to detect the changes in pH brought about by the production of ammonia and CO₂ would have to be employed. The composition may also comprise uricase which digests uric acid and does produce an electrochemical substance. In a further embodiment, the composition may also comprise means to detect Cystatin C and albumin.

The enzymes of the composition may be from any source, providing they have creatininase and/or creatinase and/or sarcosine oxidase activity. The enzymes may be wildtype enzymes, i.e. enzymes with a polypeptide sequence that naturally occurs in a particular organisms. In other embodiments one or more of the enzymes may have a non-natural sequence, for example they may have mutations compared to a naturally occurring sequence. For instance, the enzymes may have deliberate mutations to increase their activity or specificity, for example.

In one embodiment the creatininase and/or creatinase and/or sarcosine oxidase has at least 20% identity or homology to a naturally occurring creatininase and/or creatinase and/or sarcosine oxidase enzyme, for example has at last 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 92%, or at least 94%, or at least 96%, or at least 98%, or at least 99%, or 100% identity or homology to a naturally occurring creatininase and/or creatinase and/or sarcosine oxidase enzyme.

The enzymes of the composition may have any sequence provided that they are capable of catalysing the required reactions i.e. creatininase converts creatinine to creatine; creatinase converts creatine into sarcosine and urea; and sarcosine oxidase converts sarcosine into glycine, formaldehyde and hydrogen peroxide.

The enzymes of the composition may be recombinant proteins or may be synthetic proteins.

In one embodiment, the creatininase is from Sorachim catalogue number CNH-311; and/or the creatinase is from Sorachim catalogue number CRH-211; and/or the sarcosine oxidase is from Sorachim catalogue number SAO-351.

In one embodiment it is considered that the relative ratios of the enzymes in the three-enzyme system is important in producing an optimised reaction mix. The skilled person will appreciate that in any reaction, there is a rate limiting step. Without wishing to be bound by any theory, the inventors consider that in the three-enzyme system described herein, the sarcosine oxidase enzyme is the rate limiting step. The skilled person will therefore appreciate that no matter how much of the creatininase and creatinase is added to the reaction, in one embodiment the rate of production of hydrogen peroxide will be limited by the amount of sarcosine oxidase.

In a further embodiment it will be appreciated that the actual physical amount of enzyme is important. For instance, too little enzyme, even when the enzymes are in the most appropriate ratios, will not produce a sufficient amount of hydrogen peroxide for detection at the electrode, for instance. There is considered to be no limit to the upper end of the amount of enzyme that may be added, though the skilled person will appreciate that excess enzyme is wasteful and incurs unnecessary expense.

Accordingly, in one embodiment, it is considered that, in the final mixed solution that results from the mixing of the dialysate which contains the creatinine and the enzyme composition of the present invention, the concentration of creatininase should be more than about 50 U/ml, for example more than about 75 U/ml, for example more than about 100 U/ml, for example more than about 125 U/ml, for example more than about 150 U/ml, for example more than about 175 U/ml, for example more than about 200 U/ml, for example more than about 250 U/ml, for example more than about 300 U/ml, for example more than about 325 U/ml, for example more than about 350 U/ml, for example more than about 375 U/ml, for example more than about 400 U/ml, for example more than about 425 U/ml, for example more than about 450 U/ml, for example more than about 475 U/ml, for example more than about 500 U/ml, for example more than about 525 U/ml, for example more than about 550 U/ml, for example more than about 575 U/ml, for example more than about 600 U/ml, for example more than about 625 U/ml, for example more than about 650 U/ml, for example more than about 675 U/ml, for example more than about 800 U/ml, for example more than about 825 U/ml, for example more than about 850 U/ml, for example more than about 875 U/ml, for example more than about 900 U/ml, for example more than about 925 U/ml, for example more than about 950 U/ml, for example more than about 975 U/ml, for example more than about 1000 U/ml. The skilled person will be able to determine an appropriate starting concentration of creatininase in the composition of the invention to allow the required final concentration in the reaction mix.

In the same or alternative embodiment, it is considered that, in the final mixed solution that results from the mixing of the dialysate which contains the creatinine and the enzyme composition of the present invention, the concentration of creatinase should be more than about 50 U/ml, for example more than about 75 U/ml, for example more than about 100 U/ml, for example more than about 125 U/ml, for example more than about 150 U/ml, for example more than about 175 U/ml, for example more than about 200 U/ml, for example more than about 250 U/ml, for example more than about 300 U/ml, for example more than about 325 U/ml, for example more than about 350 U/ml, for example more than about 375 U/ml, for example more than about 400 U/ml, for example more than about 425 U/ml, for example more than about 450 U/ml, for example more than about 475 U/ml, for example more than about 500 U/ml, for example more than about 525 U/ml, for example more than about 550 U/ml, for example more than about 575 U/ml, for example more than about 600 U/ml, for example more than about 625 U/ml, for example more than about 650 U/ml, for example more than about 675 U/ml, for example more than about 800 U/ml, for example more than about 825 U/ml, for example more than about 850 U/ml, for example more than about 875 U/ml, for example more than about 900 U/ml, for example more than about 925 U/ml, for example more than about 950 U/ml, for example more than about 975 U/ml, for example more than about 1000 U/ml. The skilled person will be able to determine an appropriate starting concentration of creatinase in the composition of the invention to allow the required final concentration in the reaction mix.

In the same or alternative embodiment, it is considered that, in the final mixed solution that results from the mixing of the dialysate which contains the creatinine and the enzyme composition of the present invention, the concentration of sarcosine oxidase should be more than about 10 U/ml, for example more than about 15 U/ml, for example more than about 20 U/ml, for example more than about 25 U/ml, for example more than about 30 U/ml, for example more than about 35 U/ml, for example more than about 40 U/ml, for example more than about 45 U/ml, for example more than about 50 U/ml, for example more than about 55 U/ml, for example more than about 60 U/ml, for example more than about 65 U/ml, for example more than about 70 U/ml. Preferably the amount of sarcosine oxidase in the final mixed solution that results from the mixing of the dialysate which contains the creatinine and the enzyme composition of the present invention is at least 30 U/ml. The skilled person will be able to determine an appropriate starting concentration of sarcosine oxidase in the composition of the invention to allow the required final concentration in the reaction mix.

The skilled person will appreciate that the amount of each enzyme required, and in particular the amount of the sarcosine oxidase enzyme which is considered to be rate limiting, will depend on a number of factors. For instance, the anticipated amount of creatinine to be detected will influence the amount of enzyme required. Accordingly in one embodiment the amount of each enzyme used in the reaction to determine the amount of creatinine is adjusted according to the amount of creatinine in the sample.

Preferably the final mixed solution that results from the mixing of the dialysate which contains the creatinine and the enzyme composition of the present invention comprises at least 300 U/ml creatininase.

Preferably the final mixed solution that results from the mixing of the dialysate which contains the creatinine and the enzyme composition of the present invention comprises at least 120 U/ml creatinase.

Preferably the final mixed solution that results from the mixing of the dialysate which contains the creatinine and the enzyme composition of the present invention comprises at least 15 U/ml sarcosine oxidase.

In one embodiment the final mixed solution that results from the mixing of the dialysate which contains the creatinine and the enzyme composition of the present invention comprises at least 300 U/ml creatininase, 120 U/ml creatinase and at least 15 U/ml sarcosine oxidase.

Although a final mixed solution resulting from the mixing of the dialysate which contains the creatinine and the enzyme composition of the present invention which comprises less than 300 U/ml creatininase, and/or less than 120 U/ml creatinase, and/or less than 15 U/ml sarcosine oxidase is still considered to produce a useful reaction, concentrations of enzymes above these values are considered to give an even greater improvement in the reaction of creatinine to the final detectable hydrogen peroxide.

The skilled person will appreciate that the amount of each enzyme required will also depend on the length of time that the reaction is allowed to progress for before detection of the resultant hydrogen peroxide. For instance, in situations wherein the frequency of readings is not required to be high, for instance one reading an hour or more, for instance one reading every 2 hours or more, the reaction can be allowed to progress for a longer time than if a reading is required everything 0.5 seconds, or every 1 second, for instance. In the latter case a higher amount of enzyme is needed so that the reaction progresses, for example so that 90% of the creatinine is reacted with the resultant production of hydrogen peroxide (the T₉₀) whilst in the prior case a low amount of enzyme is required since the reaction can be left to progress for a longer time before detection of the hydrogen peroxide.

The inventors have optimised the reaction conditions to take account of the low physiological levels of plasma creatinine in a healthy individual and the increase levels of plasma creatinine in an individual with reduced kidney function.

In a preferred embodiment the ratio of creatininase, creatinase, and sarcosine oxidase in the final mixed solution that results from the mixing of the dialysate which contains the creatinine and the enzyme composition of the present invention is between 10:5:1 and 49:8:1 U/ml of creatininase, creatinase, and sarcosine oxidase respectively. For instance, the ratio of creatininase, creatinase, and sarcosine oxidase respectively in the final mixed solution that results from the mixing of the dialysate which contains the creatinine and the enzyme composition of the present invention may be any suitable ratio, for example may be 10:5:1, or 15:5:1, or 20:5:1, or 25:5:1, or 30:5:1, or 35:5:1, or 40:5:1, or 45:5:1, or 10:10:1, or 15:10:1, or 20:10:1, or 25:10:1, or 30:10:1, or 35:10:1, or 40:10:1, or 45:10:1, or 50:10:1, or 10:15:1, or 15:15:1, or 20:10:1, or 25:15:1, or 30:15:1, or 35:15:1, or 40:15:1, or 45:15:1, or 50:15:1, or 10:20:1, or 15:20:1, or 20:20:1, or 25:20:1, or 30:20:1, or 35:20:1, or 40:20:1, or 45:20:1, or 50:20:1, or 10:25:1, or 15:25:1, or 20:25:1, or 25:25:1, or 30:25:1, or 35:25:1, or 40:25:1, or 45:25:1, or 50:25:1, or 10:30:1, or 15:30:1, or 20:30:1, or 25:30:1, or 30:30:1, or 35:30:1, or 40:30:1, or 45:30:1, or 50:30:1, or 10:35:1, or 15:35:1, or 20:35:1, or 25:35:1, or 30:35:1, or 35:35:1, or 40:35:1, or 45:35:1, or 50:35:1, or 10:40:1, or 15:40:1, or 20:40:1, or 25:40:1, or 30:40:1, or 35:40:1, or 40:40:1, or 45:40:1, or 50:40:1, 10:45:1, or 15:45:1, or 20:45:1, or 25:45:1, or 30:45:1, or 35:45:1, or 40:45:1, or 45:45:1, or 50:45:1; or 10:50:1, or 15:50:1, or 20:50:1, or 25:50:1, or 30:50:1, or 35:50:1, or 40:50:1, or 45:50:1, or 50:50:1, for example.

As discussed above it is considered to be preferable if the amount of sarcosine oxidase in the final mixed solution that results from the mixing of the dialysate which contains the creatinine and the enzyme composition of the present invention is more than about 10 U/ml and preferably at least 30 U/ml. This is considered to allow sufficient amounts of this enzyme to give a reliable signal for the low levels of creatinine found in healthy subjects, and is able to detect creatinine levels as low as 4.3 uM with an improvement in the recovery of creatinine from the sample expected to increase the sensitivity to as low as 2 uM. The serum creatinine concentration of a healthy individual ranges from between 60 uM to 120 uM, so it is clear that the sensitivity of the claimed invention is suitably high to allow an accurate determination of the serum creatinine levels.

In a particular embodiment, the combination of particular pH of the buffer and/or the type of buffer of the composition and/or the ratio of the enzymes and/or actual amounts of each of the enzymes is considered to provide a particularly effective set of reaction conditions. For instance, in one embodiment the composition comprises the enzymes and buffer such that in the final mixed solution that results from the mixing of the dialysate which contains the creatinine and the enzyme composition there is 600 U/ml or creatininase, 300 U/ml of creatinase and 60 U/ml of sarcosine oxidase. In a further embodiment the composition comprises the enzymes and buffer such that in the final mixed solution that results from the mixing of the dialysate which contains the creatinine and the enzyme composition there is 600 U/ml or creatininase, 300 U/ml of creatinase and 60 U/ml of sarcosine oxidase at a pH of 8.5.

In one embodiment the composition comprises any two or more of creatininase, creatinase and/or sarcosine oxidase, and other components such as the buffers described herein such that the T₉₀ of a reaction comprising 100 uM creatinine is less than 10 minutes for example is less than 9.5 minutes, for example is less than 9 minutes, for example is less than 8.5 minutes, for example is less than 8 minutes, for example is less than 7.5 minutes, for example is less than 7 minutes, for example is less than 6.5 minutes, for example is less than 6 minutes, for example is less than 5.5 minutes, for example is less than 5 minutes, for example is less than 250 seconds, for example is less than 225 seconds, for example is less than 200 seconds, for example is less than 190 seconds, for example is less than 180 seconds, for example is less than 170 seconds, for example is less than 160 seconds, for example is less than 150 seconds, for example is less than 140 seconds, for example is less than 130 seconds, for example is less than 120 seconds, for example is less than 110 seconds, for example is less than 100 seconds, for example is less than 90 seconds, for example is less than 80 seconds, for example is less than 70 seconds, for example is less than 60 seconds, for example is less than 50 seconds, for example is less than 40 seconds, for example is less than 30 seconds, for example is less than 20 seconds for example is less than 10 seconds. In one embodiment the T90 is 195 seconds or less. In another embodiment the T90 is 154 seconds or less. In yet another embodiment the T90 is 135 seconds or less. The skilled person will be able to determine suitable parameters, and examples are given in the Examples.

It will be apparent to the skilled person that the composition of the invention may comprise additional components or agents, for instance other agents useful in determining the health of a subject. For instance, the composition may comprise means to detect the level of urea, for instance urease and/or uricase. The composition may also comprise means to detect Cystatin C and albumin.

It will be clear that the sensitive, optimised composition detailed herein can be used to detect the level of creatinine in a number of ways.

Accordingly, a further aspect provides a sensor system comprising creatininase and/or creatinase and/or sarcosine oxidase and at least a first sensor.

In one embodiment the creatininase and/or creatinase and/or sarcosine oxidase are provided as a composition according to the invention described herein. In another embodiment the three enzymes are provided separately and are added to the reaction mix sequentially. It will be appreciated that although the above preferences relate to a composition comprising two or more of creatininase, creatinase and/or sarcosine oxidase, the optimal reaction conditions apply to any reaction in which the three enzymes take part. For instance, the composition of the invention comprising creatininase and creatinase may be used along side a separate composition or aliquot of sarcosine oxidase. The preferred conditions, for instance a buffer that is not PBS and/or a buffer that has a pH of 8.5 still apply. Accordingly, the above conditions and preferences described in relation to the composition of the invention also apply to the situation in which all three enzymes are supplied separately, and are for instance introduced to the reaction vessel sequentially.

The sensor system therefore can include at least the following various situations:

a composition comprising creatininase and creatinase, and not sarcosine oxidase;

a composition comprising creatininase and sarcosine oxidase, but not creatinase;

a composition comprising creatinase and sarcosine oxidase, but not creatininase;

a composition comprising creatininase and creatinase, with sarcosine oxidase supplied separately;

a composition comprising creatininase and sarcosine oxidase, with creatinase supplied separately;

a composition comprising creatinase and sarcosine oxidase, with creatininase supplied separately; and

the separate supply of creatininase, creatinase and sarcosine oxidase—not as part of any same composition.

As discussed above, in one embodiment the sensor system may also include one or more buffers as described herein to allow the reaction to proceed under the optimal conditions.

The three-enzyme system produces both urea and hydrogen peroxide, both of which are detectable.

It is considered to be advantageous to detect the hydrogen peroxide generated by the sarcosine oxidase enzyme. This allows sensitive electrochemical detection by, for instance, an amperometric sensor. The hydrogen peroxide may also be detected potentiometrically, using specialiased membranes. Alternatively, hydrogen peroxide may be detected optically by the use of enzymes, for example horseradish peroxidase and a dye molecule. Accordingly, the system may comprise an amperometric sensor and/or specialised membranes for potentiometric sensing and/or a further enzyme, for instance horseradish peroxidase and a dye molecule.

Preferably, the system comprises at least an amperometric sensor. Amperometric sensors are well known in the art.

It is considered to be useful if the detection electrode is protected by one of a number of agents. Such agents are known to those in the art and include mPD, olyphenol and nafion and para-phenylenediamine (pPD). These agents are considered to prevent unwanted molecules from accessing the electrode, whilst allowing hydrogen peroxide through. In one embodiment the detection electrode is made from platinum, which is considered to be the most suitable material for hydrogen peroxide electrochemistry. In another embodiment the detection electrode may be a platinum-sputtered silicone needle or a carbon nanotube, for example.

The sensor system can be used to detect creatinine in any sample, for example a sample obtained from a subject, for example a sample taken from the blood, plasma, urine, tissue fluid or cerebrospinal fluid; or a sample taken from, for instance, a perfused kidney, for instance a sample of perfusate from a perfused kidney intended for organ transplantation.

The sensor system can also be used with any volume of sample. Advantageously, the composition and system of the invention is suitable for use with microfluidics, for example a microfluidic circuit and/or a microfluidic device and/or a microfluidic probe. Accordingly, in one embodiment the system comprises a microfluidic circuit and/or a microfluidic device and/or a microfluidic probe. Microfluidic circuits, microfluidic devices and microfluidic probes are well known in the art and particular examples are detailed in the Examples.

In one embodiment the system comprises a sampling probe, such as a microfluidic probe. Suitable microfluidic probes are known in the art and include Brain CMA-70 (from MDialysis); a Freeflap CMA-70 (from MDialysis); a MAB9.14.2 (Microbiotech SE); MAB6.14.2 (Microbiotech SE); MAB11.35.4 (Microbiotech SE) or the number 67 intravenous microdialysis catheter from MDialysis.

In another embodiment, the system also comprises a zone in which the sample, for example the microdialysate, can be mixed with the composition of the invention or with the creatininase and/or creatinase and/or sarcosine oxidase to generate hydrogen peroxide. The system also comprises, in another embodiment, a section in which the hydrogen peroxide is detected, for example by an amperometric sensor.

In one embodiment, the system also comprises a continuous flow system.

In another embodiment the system does not comprise a continuous flow system.

In a further embodiment the system comprises a means for maintaining a steady flow. This is considered to be advantageous when the real-time or continuous monitoring of kidney function is required.

In another embodiment the system does not comprise means for maintaining a steady flow. For example the system may be for use in a linear flow assay system. Such a system may be considered to be suitable for use in the home. For example, in one embodiment the system comprises the sensing reagent as described herein, for instance with a suitable buffer at a suitable pH, and a sensor, for example an electrochemical sensor wherein the system is used in a single-shot point of care situation or home test kit or device where a sample, for instance blood, is mixed with the sensing reagent and buffer if present, allowed to mix and react and then the resultant hydrogen peroxide is sensed with the sensor, for example an amperometric sensor or potentiometric sensing and/or an enzymatic sensor, for instance horseradish peroxidase and a dye molecule which allows a visual readout of the degree of kidney function.

It will be appreciated that the system may also comprise calibration standards. Accordingly in one embodiment the system may comprise means of switching between a calibration stream and a sample stream. In another embodiment the system may comprise calibration standards in the form of a parallel stream. This latter embodiment is considered to be particularly useful in the context of a home-system or point-of-care system.

The system may also comprise means to take a sample from a patient, for example from the blood, urine, plasma, tissue fluid or cerebrospinal fluid, though any suitable sample is suitable for use with the invention. The system may also comprise means to take a sample from a closed-loop isolated perfused organ, for example a kidney.

In one embodiment the sample is a dialysate, for example a microdialysate.

As discussed above the skilled person will appreciate that the closer to completion the reaction is allowed to proceed, the higher the sensitivity. The skilled person will also appreciate that there may be a compromise point that is reached between desired sensitivity and reaction time. To decrease the time taken to completion or near completion, the amount of enzyme can be increased.

In one embodiment the sensor system is arranged such that the sensing reagent is added to the sample prior to contacting the sample with the sensor. In this way the enzymes can produce an appreciable level of hydrogen peroxide prior to sensing. In a preferred embodiment the reaction has gone to completion prior to sensing, or has reached at least 95% completion prior to sensing, or has reached at least 90% completion prior to sensing, or has reached at least 85% completion prior to sensing, or has reached at least 80% completion prior to sensing, or has reached at least 75% completion prior to sensing, or has reached at least 70% completion prior to sensing, or has reached at least 65% completion prior to sensing, or has reached at least 60% completion prior to sensing, or has reached at least 55% completion prior to sensing, or has reached at least 50% completion prior to sensing, or has reached at least 45% completion prior to sensing, or has reached at least 40% completion prior to sensing.

In one embodiment the sensor system is arranged such that the sensing reagent (which as discussed above may be a composition comprising two or more of creatininase, creatinase and/or sarcosine oxidase, or may be all three enzymes in separate aliquots) is added to the sample prior to contact with the sensor, for example is arranged such that there is more than 10 minutes between adding the sensing reagent to the sample and contact with the sensor. In one embodiment the sensor system is arranged such that there is more than 10 minutes between adding the enzymes or composition of the invention to the sample and contact with the sensor, for example is more than 9.5 minutes, for example is more than 9 minutes, for example is more than 8.5 minutes, for example is more than 8 minutes, for example is more than 7.5 minutes, for example is more than 7 minutes, for example is more than 6.5 minutes, for example is more than 6 minutes, for example is more than 5.5 minutes, for example is more than 5 minutes, for example is more than 250 seconds, for example is more than 225 seconds, for example is more than 200 seconds, for example is more than 190 seconds, for example is more than 180 seconds, for example is more than 170 seconds, for example is more than 160 seconds, for example is more than 150 seconds, for example is more than 140 seconds, for example is more than 130 seconds, for example is more than 120 seconds, for example is more than 110 seconds, for example is more than 100 seconds, for example is more than 90 seconds, for example is more than 80 seconds, for example is more than 70 seconds, for example is more than 60 seconds, for example is more than 50 seconds, for example is more than 40 seconds, for example is more than 30 seconds, for example is more than 20 seconds for example is more than 10 seconds, for example is more than 5 seconds, for example is more than 2 seconds, for example is more than 1 second.

In one embodiment the sensor system is arranged such that there is less than 10 minutes between adding the enzymes or composition of the invention to the sample and contact with the sensor, for example is less than 9.5 minutes, for example is less than 9 minutes, for example is less than 8.5 minutes, for example is less than 8 minutes, for example is less than 7.5 minutes, for example is less than 7 minutes, for example is less than 6.5 minutes, for example is less than 6 minutes, for example is less than 5.5 minutes, for example is less than 5 minutes, for example is less than 250 seconds, for example is less than 225 seconds, for example is less than 200 seconds, for example is less than 190 seconds, for example is less than 180 seconds, for example is less than 170 seconds, for example is less than 160 seconds, for example is less than 150 seconds, for example is less than 140 seconds, for example is less than 130 seconds, for example is less than 120 seconds, for example is less than 110 seconds, for example is less than 100 seconds, for example is less than 90 seconds, for example is less than 80 seconds, for example is less than 70 seconds, for example is less than 60 seconds, for example is less than 50 seconds, for example is less than 40 seconds, for example is less than 30 seconds, for example is less than 20 seconds for example is less than 10 seconds, for example is less than 5 seconds, for example is less than 2 seconds, for example is less than 1 second.

The flow rate of the perfusate and the composition of the invention or the sensing reagent (in which the three enzymes are delivered sequentially rather than contemporaneously) affect the composition of the resultant reaction mix. The skilled person will be able to determine appropriate flow rates to achieve the optimal reaction mix, as described herein. In one embodiment the sensor system is arranged so that the perfusate flow rate is between 0.1-10 ul/min, for example at least 0.1 ul/min, for example at least 0.25 ul/min, for example at least 0.5 ul/min, for example at least 0.75 ul/min, for example at least 1.0 ul/min, for example at least 1.25 ul/min, for example at least 1.5 ul/min, for example at least 1.75 ul/min, for example at least 2.0 ul/min, for example at least 2.25 ul/min, for example at least 2.5 ul/min, for example at least 2.75 ul/min, for example at least 3.0 ul/min, for example at least 3.25I/min, for example at least 3.5 ul/min, for example at least 3.75 ul/min, for example at least 4.0 ul/min, for example at least 4.25 ul/min, for example at least 4.5 ul/min, for example at least 4.75 ul/min, for example at least 5.0 ul/min, for example at least 5.25 ul/min, for example at least 5.5 ul/min, for example at least 5.75 ul/min, for example at least 6.0 ul/min, for example at least 6.25 ul/min, for example at least 6.5 ul/min, for example at least 6.75 ul/min, for example at least 7.0 ul/min, for example at least 7.25 ul/min, for example at least 7.75 ul/min, for example at least 8.0 ul/min, for example at least 8.25 ul/min, for example at least 8.5 ul/min, for example at least 8.75 ul/min, for example at least 9.0 ul/min, for example at least 9.25 ul/min, for example at least 9.5 ul/min, for example at least 9.75 ul/min, for example at least 10.0 ul/min.

In a preferred embodiment the flow rate of the perfusate is between 1 ul/min and 2 ul/min. in one embodiment the flow rate of the perfusate is 1 ul/min. In another embodiment the flow rate is 2 ul/min.

It will be appreciated that the flow rate of the enzyme depends on the concertation of the enzymes and the final desired concentrations in the reaction mix.

For instance, where the composition comprises creatininase, creatinase and sarcosine oxidase in the amounts 600 U/ml, 200 U/ml and 60 U/ml respectively, in a buffer at pH 8.5, the enzyme mix will be added to create a final volumetric ratio of between 1:1 and 1:10 of enzyme mix/composition of the invention:dialysate. In one embodiment the enzyme mix/composition of the invention is added to created a final volumetric ratio of 1:4—enzyme mix/composition of the invention:dialysate. In such an embodiment the flow rate of the perfusate may be 2 ul/min and the flow rate of the enzymes/composition of the invention may be 0.5 ul/min.

The skilled person will appreciate that if the concentration of the enzymes increases or decreases, then the ratio of enzyme solution to perfusate will change.

It will be appreciated that the reaction between sarcosine oxidase and sarcosine requires oxygen. Accordingly, in one embodiment the system comprises means to increase the amount of oxygen in the reaction mix. In one embodiment the means increase the amount of oxygen in the reaction solution to more than 10 uM or more. For instance, the means to increase the amount of oxygen in the reaction solution result in an oxygen concentration of more than 25 uM, for example more than 50 uM, for example more than 75 uM or more than 100 uM, for example more than 125 uM, for example more than 150 uM, for example more than 175 uM, for example more than 200 uM, for example more than 225 uM, for example more than 250 uM, for example more than 275 uM, for example more than 300 uM, for example more than 325 uM, for example more than 350 uM, for example more than 375 uM, for example more than 400 uM, for example more than 425 uM, for example more than 450 uM, for example more than 475 uM, for example more than 500 uM. In one embodiment the concentration of oxygen is between about 200 uM to 250 uM. The engineering toolbox on http://www.engineeringtoolbox.com/oxygen-solubility-water-d_841.html gives the range of oxygen concentrations in saline solutions at normal pressures—225 umol O2 in 35% saline water at 1 atmosphere of pressure.

The amount of oxygen in the reaction mix may be increased by a number of ways, all are which may be included in the system of the invention. For instance, the system may include a mixer, that in turn includes baffles or serpentine zones or for instance anything that increases the mixing of solutions. The mixer may be made out of a highly permeable material such as PDMS, or have multiple mixing stages connected by Teflon tubing to allow the depleted oxygen levels to ‘re-charge’. The skilled person will appreciate that permeability can be achieved by either the material's intrinsic permeability or by being thin-walled, or a large surface area, or a combination of all. In one embodiment multiple means of increasing the oxygen content of the reaction mix are used, for instance multiple mixers and multiple connections made of Teflon. In a further embodiment the means of increasing the oxygen content, or the multiple means, are in a pressurised container.

As discussed above, the optimisation of the reaction conditions allows a very sensitive and accurate determination of the level of creatinine. In one embodiment therefore the system is capable of detecting creatinine at a level of 4 uM or less in solution, for example can detect 2 uM or less or 1 uM or less creatinine. In another embodiment, the sensor system can detect a change in creatinine of less than 1 uM, or less than 2 uM or less than 3 uM or less than 4 uM, or less than 5 uM or less than 7.5 uM or less than 10 uM, for example against a background level of creatinine of between 40 uM to 120 uM.

In a preferred embodiment, the sensor system comprises means for collecting data from the sensor. Such means are well known in the art, one example of which is the PowerLab/4SP.

The sensor system may also comprise, in some embodiments, a wireless transmitting means for transmitting the data, for example a Bluetooth transmitter or other wireless transmitter.

The sensor system may also comprise, in some embodiments means for data analysis, for example a computer or wearable device. In one embodiment the means for data analysis calculates the estimated glomerular filtration rate (eGFR). In a preferred embodiment the sensor system comprises a wireless transmitting means, a means for data analysis, and a means for receiving the wirelessly transmitted data.

In one embodiment the system also comprises at least one waste collection receptacle. For instance, one embodiment of the invention is a real-time monitor which may or may not be ambulatory. For ease of use, particularly in the long-term setting and/or home setting, the micodialysate, following reaction and sensing the hydrogen peroxide is considered to be waste. A preferred embodiment sees this waste product being deposited in a waste receptacle. The receptacle is preferably very small, for instance with a combined flow rate of sample/sensing reagent of 3 ul/min, a 24 hour period would produce 4.3 ml of waste. Accordingly in one embodiment the waste receptacle has a volume of less than 10 ml, for instance less than 9.5 ml, for instance less than 9 ml, for instance less than 8.5 ml, for instance less than 8 ml, for instance less than 7.5 ml, for instance less than 7 ml, for instance less than 6.5 ml, for instance less than 6 ml, for instance less than 5.5 ml, for instance less than 5 ml, for instance less than 4.5 ml, for instance less than 4 ml, for instance less than 3.5 ml, for instance less than 3 ml, for instance less than 2.5 ml, for instance less than 2 ml, for instance less than 1.5 ml, for instance less than 1 ml, for instance less than 0.5 ml, for instance less than 0.25 ml.

It will be appreciated that such a waste receptacle has uses outside the scope of the present invention and may be useful in the context of any microdialsysis treatment or analysis, or for use with other ambulatory devices.

In one embodiment the sensor system is ambulatory. For instance the sensor system may be completely independent on large machinery which the subject has to be connected, or may only require connection to such a machine for a short period of time.

The sensor system described herein allows the accurate determination of the real-time function of the kidney. As discussed, this information can be used to inform the clinician whether to being or halt treatment with a particular agent, for example a drug, or otherwise adjust a drug dosage. In one embodiment the sensor system comprises means to deliver a drug, such as a drug pump. In a preferred embodiment the sensor system comprises means to automatically adjust the working of the drug pump, i.e. the amount of drug delivered, based on the calculated creatinine level/creatinine clearance rate/glomerular filtration rate. In a preferred embodiment the determination of the amount of drug required is done automatically and without the intervention of the clinician. Such an embodiment is considered to be particularly useful in situations wherein a subject with reduced kidney function or who is at risk of having impaired kidney function uses the sensor system at home to monitor kidney function and administer the appropriate amount of relevant drug.

Examples of drugs or agents which would benefit from having their administration modulated using the system of the invention include all renally cleared drugs particularly those which may promote or damage renal clearance or whose bioactivity is dependent upon clearance rates. Such agents include contrast agents for imaging studies, whilst examples of relevant drugs include immunosuppressants; chemotherapy agents such as platinum agents; antimicrobials such as the glycopeptides vancomycin and teicoplanin, and penicillin; and opioid analgesics such as morphine, diamorphine and codeine.

Such an approach allows the dosage of the drug to be personalised based on actual renal clearance measurements in real time rather than estimated clearance measurements, based on a sample taken some time, for instance some hours, prior to the results becoming known.

The composition or sensor system of the invention can be used to monitor the steady-state level of creatinine in a subject. Any rise in this level may indicate that kidney function is becoming impaired. A decrease in this level may also indicate that other clinical intervention is required. As such, a read-out of the steady-state level of creatinine is considered to be useful.

However, to obtain a “live” reading of the GFR, in one embodiment the subject is administered creatinine and/or creatine and/or sarcosine, in order to determine the clearance rate of this artificially induced creatinine spike, and which tests the ability of the kidney to clear it from the blood. Such a method is considered to be advantageous, since it is not susceptible to factors which may affect the steady-state creatinine levels. For instance a high creatinine reading may be due to increased production of creatinine and not due to decreased kidney function. Agents within the sample may interfere with the assay, or the readings may be affected by decreased tubular secretion of creatinine. An increase in serum creatinine can also be attributed to increased ingestion of cooked meat (which contains creatinine converted from creatine by the heat from cooking) or excessive intake of protein and creatine supplements, taken to enhance athletic performance. Intense exercise can increase creatinine by increasing muscle breakdown. Several medications and chromogens can interfere with the assay. Creatinine secretion by the tubules can be blocked by some medications, again increasing measured creatinine.

The sensor system of the invention therefore may also comprise means to administer creatinine and/or creatine and/or sarcosine to the subject, for example at regular intervals. Any amount of creatinine may be administered. In one embodiment the amount of creatinine administered is sufficient to increase the baseline level by between 10% to 250%, for example between 20% and 230%, for example between 30% and 210%, for example between 40% and 200%, for example between 50% and 190%, for example between 60% and 180%, for example between 70% and 170%, for example between 80% and 160%, for example between 90% and 150%, for example between 100% and 140%, for example between 110% and 130%, for example 120%.

It is considered to be particularly useful if the amount of creatinine administered is sufficient to increase the level by double their baseline creatinine level.

The skilled person will appreciate that a subject with severely impaired kidney function will struggle to clear even a small amount of exogenously administered creatinine, whilst a subject with healthy kidneys will be able to clear a large amount of exogenously administered creatinine relatively quickly. The skilled person will be able to determine the appropriate amount of creatinine to administer to the subject to allow the required analysis to be made.

As discussed above, in a preferred embodiment the creatinine, creatine and/or sarcosine is administered automatically and without the intervention of the clinician. Such an embodiment is considered to be particularly useful in situations wherein a subject with reduced kidney function or who is at risk of having impaired kidney function uses the sensor system at home to monitor kidney function and administer the appropriate amount of relevant drug.

It will be appreciated that there may be some background level of hydrogen peroxide generated by endogenous creatine and sarcosine, i.e. not directly derived from creatinine. To improve the accuracy of the determination of the level of creatinine, the skilled person may take account of these background levels. In one embodiment the sensor system is arranged such that there comprises a second sensor and a second means to obtain a second sample. In this embodiment the second sample is contacted with a second sensing reagent that comprises creatinase and sarcosine oxidase (i.e. no creatininase) prior to detection at the second sensor. As discussed above, the enzyme concentration, ratio and time allowed for the reaction to proceed may all be optimised to provide the highest sensitivity. In one embodiment the sensor system is arranged such that there is more than 10 minutes between adding the sensing reagent to the second sample and contact with the second sensor. In one embodiment the sensor system is arranged such that there is more than 10 minutes between adding the enzymes or composition of the invention to the sample and contact with the sensor, for example is more than 9.5 minutes, for example is more than 9 minutes, for example is more than 8.5 minutes, for example is more than 8 minutes, for example is more than 7.5 minutes, for example is more than 7 minutes, for example is more than 6.5 minutes, for example is more than 6 minutes, for example is more than 5.5 minutes, for example is more than 5 minutes, for example is more than 250 seconds, for example is more than 225 seconds, for example is more than 200 seconds, for example is more than 190 seconds, for example is more than 180 seconds, for example is more than 170 seconds, for example is more than 160 seconds, for example is more than 150 seconds, for example is more than 140 seconds, for example is more than 130 seconds, for example is more than 120 seconds, for example is more than 110 seconds, for example is more than 100 seconds, for example is more than 90 seconds, for example is more than 80 seconds, for example is more than 70 seconds, for example is more than 60 seconds, for example is more than 50 seconds, for example is more than 40 seconds, for example is more than 30 seconds, for example is more than 20 seconds for example is more than 10 seconds, for example is more than 5 seconds, for example is more than 2 seconds, for example is more than 1 second.

In one embodiment the sensor system is arranged such that there is less than 10 minutes between adding the creatinase and sarcosine oxidase to the second sample and contact with the second sensor, for example is less than 9.5 minutes, for example is less than 9 minutes, for example is less than 8.5 minutes, for example is less than 8 minutes, for example is less than 7.5 minutes, for example is less than 7 minutes, for example is less than 6.5 minutes, for example is less than 6 minutes, for example is less than 5.5 minutes, for example is less than 5 minutes, for example is less than 250 seconds, for example is less than 225 seconds, for example is less than 200 seconds, for example is less than 190 seconds, for example is less than 180 seconds, for example is less than 170 seconds, for example is less than 160 seconds, for example is less than 150 seconds, for example is less than 140 seconds, for example is less than 130 seconds, for example is less than 120 seconds, for example is less than 110 seconds, for example is less than 100 seconds, for example is less than 90 seconds, for example is less than 80 seconds, for example is less than 70 seconds, for example is less than 60 seconds, for example is less than 50 seconds, for example is less than 40 seconds, for example is less than 30 seconds, for example is less than 20 seconds for example is less than 10 seconds.

In a preferred embodiment the sensor system also comprises means to subtract the data obtained from the second sensor from the data obtained from the first sensor. In this way a true determination of the level of creatinine is obtained. However, determination of this background level of hydrogen peroxide that may be produced from endogenous creatine and sarcosine is not considered to be essential. These levels are considered to be low and generally insignificant. In addition, the present invention allows the relative amounts of creatinine to be determined and changes thereof, i.e. within a particular subject. The actual physical amount of creatinine is not considered to be as important as any relative changes in the perceived amount of creatinine following, for example, drug administration.

It is also known that tubular creatinine secretion contributes to the overall total amount of creatinine. To further correct for this, the system may be arranged so that the drug cimetidine is also administered to the subject prior to the reaction to determine creatinine levels. Cimetidine is considered to inhibit tubular secretion of creatinine. In this case the kinetics are completely first order and the amount of creatinine in the blood is dependent only on functioning nephrons.

It will be appreciated that one of the real advantages of the current invention is the realisation of the ability to monitor kidney function in real-time. Accordingly, in one embodiment, the sensor system captures data continuously. For example, where the sensor system comprises microfluidics, in one embodiment the sensor reagent of the invention is flowed continuously into a stream of microdialysate from a subject. Following an appropriate reaction time, which can be set simply by changing the length of the path that the reaction mixture has to take until it reaches the sensor (preferably via one or more mixers and/or one or more components that increase the oxygen concentration in the reaction mix, as discussed above) the amount of hydrogen peroxide is determined, the amount of creatinine in the sample is then determined and used to calculate the GFR, if required. This can be continuous and give a true real-time and continuous read of the subjects creatinine levels.

Alternatively, a continuous read of the creatinine levels may be considered to be unnecessary and data from different discrete times points may be considered to be sufficient. Although the flow of analyte may be continuous, the sampling of the data may or may not be continuous. If the sampling of the data is not continuous it still may occur sufficiently fast enough for an effective continuous stream of data. For example the reading from the sensor may be digisited at approximately 200 Hz. The sample can be digitised at as low a frequency as 10 Hz and still give an effectively continuous stream of data. The reading from the sensor may be digitised at much faster rates than 200 Hz. However, it is considered that there is a limit to the usefulness of data obtained over a particular rate. For example, the data should be obtained at a rate sufficiently high enough to rapidly detect changes in metabolite or molecule level, but perhaps not so great a rate as it generates too much non-useful data which may overpower data analysis systems. For example a reading every 10 seconds may be considered acceptable, or an average reading over every 10 seconds, providing an average of continuously obtained data.

Accordingly, in one embodiment the sensor system captures data at least every 24 hours, or at least every 22 hours, for example at least every 20 hours, for example at least every 18 hours, for example at least every 16 hours, for example at least every 14 hours, for example at least every 12 hours, for example at least every 10 hours, for example at least every 8 hours, for example at least every 6 hours, for example at least every 5 hours, for example at least every 4 hours, for example at least every 3 hours, for example at least every 2 hours for example at least every 1.5 hours, for example at least every 1 hour, for example at least every 50 minutes, for example at least every 45 minutes, for example at least every 40 minutes, for example at least every 35 minutes, for example at least every 30 minutes, for example at least very 25 minutes, for example at least every 20 minutes, for example at least every 15 minutes, for example at least every 10 minutes, for example at least every 5 minutes, for example at least every 2 minutes, for example at least every 1.5 minutes, for example at least every 60 seconds, for example at least every 45 seconds, for example at least every 30 seconds, for example at least every 15 seconds, for example at least every 10 seconds, for example at least every 5 seconds, for example at least every 2 seconds, for example at least every 1 second for example at least every 0.5 seconds.

In one embodiment the data obtained is an average reading of a particular interval, for instance is an average reading across at least every 24 hours, for example at least every 22 hours, for example at least every 20 hours, for example at least every 18 hours, for example at least every 16 hours, for example at least every 14 hours, for example at least every 12 hours, for example at least every 10 hours, for example at least every 8 hours, for example at least every 6 hours, for example at least every 5 hours, for example at least every 4 hours, for example at least every 3 hours, for example at least every 2 hours for example at least every 1.5 hours, for example at least every 1 hour, for example at least every 50 minutes, for example at least every 45 minutes, for example at least every 40 minutes, for example at least every 35 minutes, for example at least every 30 minutes, for example at least every 25 minutes, for example at least every 20 minutes, for example at least every 15 minutes, for example at least every 10 minutes, for example at least every 5 minutes, for example at least every 2 minutes, for example at least every 1.5 minutes, for example at least every 60 seconds, for example at least every 45 seconds, for example at least every 30 seconds, for example at least every 15 seconds, for example at least every 10 seconds, for example at least every 5 seconds, for example at least every 2 seconds, for example at least every 1 second for example at least every 0.5 seconds.

Current clinical practice is to analyse kidney function three times a day. Accordingly in one embodiment the sensor system captures data three times a day, for instance every 8 hours.

Data capture may occur on a regular basis, or may be irregular. For instance data capture may occur more frequently at times of increased risk, for example following administration of a drug, and may be less frequent a times of less risk.

As discussed above, the sensor system may comprise a wireless transmitter which transmits the data to a means for data analysis. As with data capture, transmission of the data may be continuous, or may be at regular or irregular intervals. For instance the data may be transmitted at least every 24 hours, for example at least every 22 hours, for example at least every 20 hours, for example at least every 18 hours, for example at least every 16 hours, for example at least every 14 hours, for example at least every 12 hours, for example at least every 10 hours, for example at least every 8 hours, for example at least every 6 hours, for example at least every 5 hours, for example at least every 4 hours, for example at least every 3 hours, for example at least every 2 hours for example at least every 1.5 hours, for example at least every 1 hour, for example at least every 50 minutes, for example at least every 45 minutes, for example at least every 40 minutes, for example at least every 35 minutes, for example at least every 30 minutes, for example at least very 25 minutes, for example at least every 20 minutes, for example at least every 15 minutes, for example at least every 10 minutes, for example at least every 5 minutes, for example at least every 2 minutes, for example at least every 1.5 minutes, for example at least every 60 seconds, for example at least every 45 seconds, for example at least every 30 seconds, for example at least every 15 seconds, for example at least every 10 seconds, for example at least every 5 seconds, for example at least every 2 seconds, for example at least every 1 second for example at least every 0.5 seconds.

As mentioned above, current clinical practice is to analyse kidney function three times a day. Accordingly in one embodiment the sensor system transmits the data three times a day, for instance every 8 hours.

In one embodiment, it is considered to be useful to monitor the level of urea. Accordingly in one embodiment the system comprises means to determine the level of urea. For instance, it is considered to be useful if the composition of the invention also comprises urease, to allow the detection of urea, though the skilled person will appreciate that this reaction does not produce an electrochemical substance, and so the system may also comprises means to detect the changes in pH brought about by the production of ammonia and CO2. The composition of the invention may also comprise uricase which digests uric acid and does produce an electrochemical substance which can be detected using one or more sensor in the system. In a further embodiment, the system also comprises means to detect Cystatin C and albumin.

As discussed above, the invention provides various compositions comprising any two or more of creatininase, creatinase and/or sarcosine oxidase, along with various other components and parameters for optimal enzyme activity. The invention also provides a sensor system, which comprises components that are considered to be advantageous in the actual determination of the creatinine level of a subject, in addition to a sensing reagent which may be the same as the composition, or may instead comprise creatininase, creatinase and sarcosine oxidase in separate vessels for sequential use.

The invention also provides various methods of using the compositions and sensor system of the invention. Preferences for the various features of the composition, sensing reagent and sensor system of the invention discussed above also apply below.

In one embodiment, the invention provides a method for the determination of the level of creatinine in a sample from a human or animal subject, wherein the method comprises the use of the composition or sensor system of the invention. In a preferred embodiment the sample is a dialysate or a microdialysate.

The level of creatinine can be used to determine the glomerular filtration rate (GFR), accordingly, the invention also provides a method for the determination of the GFR in a human or animal subject, wherein the method comprises the use of the composition or sensor system of the invention. In a preferred embodiment the sample is a dialysate or a microdialysate.

Since the present invention uniquely allows the real-time determination of the level of creatinine, the invention also provides a method for the real-time determination of the level of creatinine, or creatinine clearance rate or GFR in a sample from a human or animal subject, wherein the method comprises the use of the composition of sensor system of the invention, optionally wherein the sample is a dialysate or a microdialysate.

Preferences for the methods include those preferences discussed above in relation to the composition of the invention or sensor system of the invention. For example, in any of the methods of the invention, in one embodiment the composition of the invention or the three separate enzymes are added prior to contacting the sample with the sensor. In another embodiment the subject is administered an amount of creatinine and the clearance rate determined. Also as discussed above the drug cimetidine may also be administered prior to the determination of the creatinine levels.

It will be apparent to the skilled person that the methods, compositions and sensor systems described herein can be used in methods of diagnosis. For instance, in one embodiment the invention provides a method for diagnosing a subject as having acute or chronic kidney disease, the method comprising determining the creatinine level and/or the creatinine clearance rate and/or the glomerular filtration rate according to any of the methods described herein.

For instance, if the steady-state level of creatinine begins the rise, then the subject may be starting to suffer from kidney damage and impaired kidney function. Additionally or alternatively if following administration of an amount of creatinine, the rate of clearance is not as fast as it was when a previous amount of creatinine was administered, then again the subject may be beginning to suffer kidney damage.

Following such a diagnosis, the method may also comprise treating the subject for acute or chronic kidney disease. This may involve stopping treatment with or reducing the dosage of a drug that is contraindicated or dangerous in acute or chronic kidney disease, or may involve stopping treatment with or reducing the dosage of a drug that has been recently administered and may be considered to be responsible for the impaired kidney function.

For example, opioid analgesics in particular morphine, diamorphine, codeine and chemotherapy agents such as the platinum agents are considered to be drugs that the administration of may be modulated following determination of kidney function using the methods of the invention. Other drugs are known in the art, for instance http://www.eastmidlandscancernetwork.nhs.uk/Library/RenalDosageAdjustments.pdf details the recommended dosage adjustment based on GFR of a number of drugs.

For example, cytarabine is completely contraindicated (Cl) with GFR below 30 ml/min. The accuracy and sensitivity of detection of creatinine levels with the present invention may allow these patients to just receive a personalised dosage of this useful drug, rather than stopping treatment altogether.

Other such drugs include antibiotics, for instance the glycopeptides vancomycin and teicoplanin, or increasing dosages of penicillins. More information can be found at: [symbol] gle.co [symbol] sa=t&rct=j&q=&esrc=s&source=web&cd=2&ved=0ahUKEwjQh5v63JDVAhVgOMAKHSnrByUQFggtMAE&url=https%3A%2F%2Fwww.nuh.nhs.uk%2Fhandlers%2Fdownloads.ashx%3Fid%3D60983&usg=AFQjCNFrkdOqgEltY0E8rSWu4GJmTbRgOQ

The invention therefore also provides a method for determining a dose of a drug to be administered to a subject, the method comprising determining the creatinine level/creatinine clearance rate/glomerular filtration rate at least prior to administration of a drug and at least after administration of the drug, optionally further comprising comparing the creatinine level/clearance rate/glomerular filtration rate prior to and after administration of the drug.

A method of adjusting the dosage of a drug such as that described herein in also considered to be useful in drug trials.

The invention also provides a method for determining a dose of a drug to be administered to a subject based on the baseline creatinine level of the subject alone. For instance, if a subject is considered to have high levels of blood creatinine, then the dosage of a drug may be reduced, or may not be administered at all.

Alternatively, the methods may also comprise maintaining or increasing the dose of the drug if the creatinine levels maintain a steady state following administration of the drug.

The above methods can be used in a method of diagnosing a subject as having chronic kidney disease. For instance, chronic kidney disease is generally diagnosed based on high levels of creatinine over a prolonged period.

| Stage | GFR* | Description | Treatment stage |
|---|---|---|---|
| 1 | 90+ | Normal kidney function but urine | Observation, control of blood pressure. |
| | | findings or structural | |
| | | abnormalities or genetic trait | |
| | | point to kidney disease | |
| 2 | 60-89 | Mildly reduced kidney function, | Observation, control of blood pressure |
| | | and other findings (as for stage 1) | and risk factors. |
| | | point to kidney disease | |
| 3A | 45-59 | Moderately reduced kidney | Observation, control of blood pressure |
| 3B | 30-44 | function | and risk factors. |
| 4 | 15-29 | Severely reduced kidney function | Planning for endstage renal failure. |
| 5 | <15 or | Very severe, or endstage kidney | Treatment choices. |
| | on | failure (sometimes | |
| | dialysis | call established renal failure) | |

In a preferred embodiment, the methods of the invention are repeated, for instance the determination of creatinine levels and GFR can be made on a continuous basis or at regular or irregular intervals, as discussed above in relation to the sensor system. The frequency that the methods should be carried out will depend on the aims and can readily be determined by the skilled person. For instance, the methods may be carried out very frequently if the subject is considered to be at risk for kidney failure, or may be carried out less frequently where the subject is not considered to be at an increased risk of kidney failure. The dosage of a drug can be adjusted regularly, or on a live real-time basis.

As discussed above, it is advantageous in some situations to administer a “spike” of creatinine and/or creatine and/or sarcosine to a subject, to allow the kinetics of the clearance of the creatinine to be observed. For instance the time it takes for creatinine levels (for example), to return to a baseline level is indicative of the function of the kidney. In this situation it is advantageous to monitor the creatinine levels (or creatine or sarcosine as the case may be) immediately after administration of the creatinine.

As discussed above, any of the methods of the invention may be performed on any type of sample from a subject, for instance a blood sample or plasma or a urine sample, or tissue fluid of cerebrospinal fluid. In one embodiment the sample is a dialysate or microdialysate, for example from any of blood, urine, tissue fluid, or cerebrospinal fluid.

It will be appreciated that the methods of the invention may be used to determine kidney function, i.e. a GFR based on the creatinine level as determined by the present invention, in isolation from reference samples, for instance reference samples of a known creatinine concentration. In one embodiment it is considered that the relative change in creatinine level or calculated GFR that is to be used to determine, for instance, whether or not a drug should be administered, or how much of a drug to administer is based only on the relative changes in kidney function of that subject. For example if the baseline creatinine level begins to increase then the subject is considered to be starting to display signs of impaired kidney function. Or if following spiking with creatinine, creatine or sarcosine the rate at which that creatinine, creatine or sarcosine is cleared is lower than the rate at which the creatinine, creatine or sarcosine was cleared in a previous test.

However, in some embodiments the determined level of creatinine, or the calculated GFR is compared to a reference sample of known creatinine concentration and so in another embodiment the invention provides a method for monitoring renal function, wherein the method comprises contacting a sample with the composition or sensing reagent as defined in any of the preceding claims, optionally wherein the method comprises determination of the concentration of creatinine in the sample, and optionally further comprises comparison to a reference sample or known reference concentration, optionally wherein the method comprises detection of the level of H₂O₂, optionally by use of an electrochemical sensor, optionally by amperometry.

It is considered that even in the absence of real-time monitoring (which due to the present invention is now possible), the present invention is considered to be useful in for example single measurements of creatinine levels, as currently used in practice. In this case comparison of the subject sample to a reference sample, or other known set of samples with which the subject sample can be compared to provide useful information, is considered to be appropriate. In one embodiment therefore the invention provides a method of determining the concentration of creatinine in a sample wherein the method comprises contacting a sample with the composition or sensing reagent as defined in any of the preceding claims. In a preferred embodiment the method comprises detection of the level of H₂O₂, for example by use of an electrochemical sensor, for example by amperometry. The method may also comprise comparison to a reference sample or known reference concentration. In a preferred embodiment all of creatininase, creatinase and sarcosine oxidase are in free solution. As discussed above the enzymes may be added separately to the subject sample, i.e. as discussed in relation to the sensing reagent above, or at least two of the enzymes may be added at the same time, for example by using the composition of the invention. In another embodiment all three enzymes are part of the same composition and so all three enzymes are added to the subject sample at the same time. Preferences for the composition discussed above, which also apply to the sensing reagent, for example choice of buffer, the buffer not being PBS, choice of pH and/or pKa all apply to this (and to all other) embodiments.

As discussed above the invention provides a method of determining the relative change in creatinine concentration wherein the method comprises contacting a sample with the composition as defined in any of the preceding claims at more than one time point, optionally wherein the method comprises comparison to a reference sample or known reference concentration.

The skilled person will appreciate that the composition, sensing system and methods described herein also have utility in the field of organ transplantation. It is considered to be beneficial if the function of a kidney that has been isolated ahead of transplantation can be monitored, such that various interventions can be put in place if the kidney function starts to decline.

The inventions have herein provided data to support the proof of concept, based on a kidney for transplantation. However it is considered that the method of adding a particular agent to a closed-loop perfusion system and monitoring the clearance or conversion of the metabolite as an indicator of organ function is widely applicable and can be applied to any organ for which there is a metabolite, the production of which or the reduction of which can be monitored.

For instance, the function of lungs for use in a transplant (i.e. lungs that have been taken from a subject) can be monitored by measuring carbon dioxide clearance. In such a situation an aliquot of carbon dioxide can be added to the perfusion system and the rate of clearance of carbon dioxide monitored. The skilled person will appreciate that the composition of the invention is not considered to be useful in the determination of the level of carbon dioxide, but the skilled person will be well aware of methods for determining the level of carbon dioxide in, for example blood, which can be directly applied to the detection of carbon dioxide in the perfusate. It is considered feasible that such an approach will work based on the work presented herein.

Similarly, the level of function of the liver may be determined by adding haeme to the closed-loop system and the production of bilirubin may be monitored, which gives a direct indicator of the function of the liver at that particular time.

For instance, in one embodiment the invention provides a method for monitoring a transplant organ, for instance a transplant organ that has been previously taken from a subject, said method comprising administrating to an isolated transplant organ an agent that is normally metabolised by a healthy organ and subsequent determination of the level of said agent or metabolite of said agent, optionally wherein said determination further comprises use of a composition, sensor system or method of the invention. Preferably the organ is in a closed-loop system.

In one embodiment the organ is a kidney, so the invention provides a method for monitoring a transplant kidney that has been previously taken from a subject, said method comprising administrating to an isolated transplant kidney creatinine, creatine or sarcosine followed by determination of the level of creatinine, creatine or sarcosine optionally wherein said determination further comprises use of a composition, sensor system or method of the invention. Preferably the kidney is in a closed-loop system.

In such a system the “spike” approach discussed above where creatinine, creatine or sarcosine is administered and then the clearance rate determined is deemed to be appropriate.

The compositions, systems and methods of the invention are also considered to be useful in the monitoring of grafts for free flap surgery. Damaged muscle tissue leaks creatinine and potassium and so the invention may be used to monitor any potential increase in creatinine that indicates that the graft is deteriorating.

For all of the methods involving transplanted organs, preferably the methods involved in determining the function of the organ, for instance the kidney, is repeated, and may be repeated regularly, for instance at least every 24 hours, for example at least every 22 hours, for example at least every 20 hours, for example at least every 18 hours, for example at least every 16 hours, for example at least every 14 hours, for example at least every 12 hours, for example at least every 10 hours, for example at least every 8 hours, for example at least every 6 hours, for example at least every 5 hours, for example at least every 4 hours, for example at least every 3 hours, for example at least every 2 hours for example at least every 1.5 hours, for example at least every 1 hour, for example at least every 50 minutes, for example at least every 45 minutes, for example at least every 40 minutes, for example at least every 35 minutes, for example at least every 30 minutes, for example at least every 25 minutes, for example at least every 20 minutes, for example at least every 15 minutes, for example at least every 10 minutes, for example at least every 5 minutes, for example at least every 2 minutes, for example at least every 1.5 minutes, for example at least every 60 seconds, for example at least every 45 seconds, for example at least every 30 seconds, for example at least every 15 seconds, for example at least every 10 seconds, for example at least every 5 seconds, for example at least every 2 seconds, for example at least every 1 second for example at least every 0.5 seconds.

In one embodiment the invention provides a method for monitoring a kidney for transplant, said method comprising perfusing the kidney and administering an amount of creatinine into the system, and determining the creatinine clearance rate using the composition and/or system of the invention.

The skilled person will clearly realise the utility of the present invention in monitoring the function of an organ in a subject following translation of that organ. The invention therefore also provides a method for monitoring kidney function in a recipient of a transplant wherein the creatinine level and/or creatinine clearance rate and/or GFR and/or kidney function is determined by use of any one or more of the composition, sensor system and/or methods described herein.

The invention also provides a method for prolonging the longevity of an isolated kidney wherein said method comprises monitoring the kidney function by use of the composition, sensor system and/or methods of any of the preceding claims, optionally wherein if kidney function begins to decline parameters such as oxygen delivery, temperature, pressure and flow rates are modified to try to increase the longevity of an isolated kidney. Research into ways to prolong transplant organ longevity is ongoing and the compositions, systems and methods of the invention are considered to be useful in checking the end-point of this research and can be used to develop drugs to improve longevity.

The compositions, sensor system and methods of the invention can be provided as various kits of parts. For instance, in one embodiment the invention provides a kit comprising:
any two or all of creatininase, creatinase and sarcosine oxidase; and/or
the composition of the invention as described herein; and/or
creatinine and/or creatine and/or sarcosine; and/or
at least one waste receptacle;
a buffer, for example a buffer as described herein, for example a buffer that is not PBS, and/or a buffer that
a microdialysis probe; and/or
at least one, preferably at least two precision pumps.

The listing or discussion of an apparently prior-published document in this specification should not necessarily be taken as an acknowledgement that the document is part of the state of the art or is common general knowledge.

Preferences and options for a given aspect, feature or parameter of the invention should, unless the context indicates otherwise, be regarded as having been disclosed in combination with any and all preferences and options for all other aspects, features and parameters of the invention. For example a method of the invention may comprise a buffer with a pH of 8.5 and a perfusate flow rate of 4 ul/min and an enzyme/composition flow rate of 0.5 ul/min.

The invention will now be exemplified by the following non-limiting examples.

### FIGURE LEGENDS

FIG. 1. Comparing 30 U/ml SAO in 10 mM PBS from pH 7.0-8.0 using a 50 μm electrode.

FIG. 2. Comparing 30 U/ml SAO in 100 mM PBS at pH 7.5, 50 mM EPPS at pH 8.0, and 50 mM borate at pH 9.0 with the 8×25 μm electrode array, demonstrating a near tripling of the current at 1 mM sarcosine in EPPS.

FIG. 3. Standardised current response profiles obtained from serial dilution experiments of 30 U/ml SAO vs. 100 uM sarcosine in various buffers, confirming the unsuitability of Tris and borate buffers for this system. Times in minutes.

FIG. 4. One of the final enzyme optimisation experiments demonstrating the normalised time evolution of the signal from the enzymatic digestion of 100 μM creatinine. All enzyme amounts in Units/ml. Note the small perturbation (*) caused by a leading edge of unbuffered NaCl at pH 3.0.

FIG. 5. Creatinine calibration curve for microdialysis at 2 μl/min, obtained by standard addition in well-stirred T1, with a parallel sampling curve from well-stirred defibrinated horse blood. Both curved obtained by auto-fitting to the Hill Equation.

FIG. 6. Testing for stability of the microdialysis sampling system over a 12 hour period. Note the spikes from the enzyme pump refilling every 40 minutes (20 μl at 0.5 μl/min). The experiment terminated just beyond the 12 hour period when the enzyme reservoir was exhausted.

FIG. 7. Testing interference by ascorbic acid (Asc), uric acid (Uric), and paracetamol (Para). Values below labels are the running total concentration. The concentration of uric acid was estimated from its maximum solubility in water at 20° C.

FIG. 8. Simulating 100 ml/min creatinine clearance with different levels of creatinine in solution. The dotted lines show the exponential curves from which the rate constants and half-lives were derived.

FIG. 9. Results of dilution experiments to simulate different degrees of renal dysfunction, from CKD1-CKD4, equivalent to creatinine clearance rates of 100 ml/min-25 ml/min.

FIG. 10. The Waters RM3 cold perfusion system configured for warm blood perfusion with an external membrane oxygenator and heat exchanger (out of frame). The microdialysis system has completed initial calibrations and is waiting for the kidney to arrive.

FIG. 11. Grey: Raw signal from the microdialysis system showing the regular electrical spikes from the RM3's pump. Black: Results of applying a Savitsky-Golay smoothing filter to the data.

FIG. 12. A time-series image of the system being tested in a real blood-perfused pig kidney. Results of the warm perfusion experiments showing an initial plateau phase followed by a steady decrease in signal magnitude following oxygenation, and the two subsequent creatinine tests.

FIG. 13. Digestion of 100 uM creatinine in NaCl at pH 3.0, 50 mM EPPS at a) pH 7.5, b) pH 8.0 and c) pH 8.5 as the running buffer.

FIG. 14. Digestion of 100 uM creatinine in NaCl at pH 3.0. a) creatininase:creatinase:sarcosine oxidase—150:300:60, b) creatininase:creatinase:sarcosine oxidase—300:300:60 and c) creatininase:creatinase:sarcosine oxidase—600:300:60.

FIG. 15. Creatine digestion in NaCl at pH 3.0, 50 mM EPPS at a) pH 7.5, b) pH 8.0 and c) pH 8.5 as running buffer.

FIG. 16. Creatine digestion in NaCl at pH 3.0. a) creatinase to SOA ratio=150:60, b) creatinase to SOA ratio=180:60 and c) creatinase to SOA ratio=300:60.

FIG. 17. a) Digestion of 100 uM sarcosine in 50 mM EPPS and b) Signal response vs time for different concentrations of creatinase vs sarcosine oxidase when digesting 100 uM creatinase.

FIG. 18. Solubility table.

FIG. 19. Summary of the experimental conditions described in the literature for the three-enzyme amperometric detection of creatinine. CA=creatininase, CI=creatinase, SO=sarcosine oxidase, normalised to U/ml in preparatory solutions, where 1 Unit catalyses the conversion of 1 pmol of substrate per minute. *U/cm² of electrode. **U/electrode. ***mg of enzyme, unable to perform conversion.

### EXAMPLES

### Example 1

### Design Requirements

The overall design concept was to create a portable, low-cost, largely turn-key, miniature system for continuously sampling and assaying normal creatinine concentrations in either the blood or urine of an isolated perfused kidney of a subject, for example a patient. This requires a system capable of detecting concentrations between 60 μm-120 μm for blood and 7-16 mM in the urine (Table 1.1, below). Note that these blood creatinine concentrations are only 1/25th- 1/150th the concentration of blood glucose, and that there are no systems presently capable of continuous real-time creatinine monitoring in a clinical setting [33].

TABLE 1.1
Normal Ranges in Blood and Urine [1, 4-6]

| | Constituent | Serum | Urine |
|---|---|---|---|
| General | Volume | ~80 ml/Kg | ~1.5 L/24 hrs |
| Properties | | Bodyweight | |
| | Osmolality | 280-295 mOsm/Kg | 450-900 mOsm/Kg |
| | pH | 7.35-7.45 | 4.5-8.0 |
| | [H⁺] | 35-45 nmol/L | 1-32,000 nmol/L |
| | Protein | 60-80 g/L | ≤150 mg/24 hrs |
| Ions | Na⁺ | 135-145 mmol/L | 40-220 mmol/24 hrs |
| | K⁺ | 3.5-5.0 mmol/L | 25-120 mmol/24 hrs |
| | Ca²⁺ | 2.0-2.5 mmol/L | 2.5-7.5 mmol/24 hrs |
| | Mg²⁺ | 0.6-0.8 mmol/L | 3-4.5 mmol/24 hrs |
| | Cl⁻ | 95-105 mmol/L | 100-250 mmol/24 hrs |
| Nitrogenous | Urea | 1.5-5 mmol/L | 420-720 mmol/24 hrs |
| Wastes | Creatinine | 60-120 μmol/L | 7-16 mmol/24 hrs |
| | Ammonia/ | 10-35 μmol/L | 20-70 mmol/24 hrs |
| | NH₄⁺ | | |
| | Uric Acid | 180-480 μmol/L | 1.4-4.4 mmol/24 hrs |
| Cells | Erythrocytes | 4-5 × 10¹²/L | 0-3/HPF† |
| | Leukocytes | 4-9 × 10⁹/L | 0-2/HPF† |
| Other | Glucose | 4-6 mmol/L | Absent |
| | Bilirubin | 2-25 μmol/L | Absent |
| | Ketones | Absent | Absent |
| | Nitrites | Absent | Absent |

†/HPF= per High Powered microscope Field

We have learned through experience is that it is important to consider the detection method for the reaction product at an early stage in the design process. Both of the reaction schemes for creatinine deiminase and the more complicated 3-step process of Tsuchida and Yoda produce species that are amenable to either electrochemical or spectrophotometric quantitation. Of these two, electrochemical methods are more suited to miniaturisation owing to the problems of optical path-lengths at small scales and the creation and stability of monochromatic light io sources required for colourimetric or absorption-based detection.

### Example 2

### Developing the Real-Time Assay System

The glucose and lactate sampling systems developed within our laboratory leverage a combination of microdialysis, microfluidics and amperometric sensing to create robust continuous-flow real-time assay systems (see for instance, WO 2016189301).

Amperometric Sensors

Our laboratory uses a potentiostat designed by a previous PhD researcher, Dr. Chu Wang [58]. This uses the OPA129 (Texas Instruments Inc., Dallas, Tex., USA) as the transimpedance amplifier, which has a maximum input bias current of 100 fA, a current noise figure of 0.1 fA/\sqrt{Hz} and a differential input impedance of 10¹³Ω. In this design, the voltage set point is applied as an inverse voltage to the counter-electrode from the PowerLab data collection system rather than a direct bias at the working electrode, so as to minimise any possible noise at the inputs of the transimpedance amplifier. The servo part of the circuit uses an OPA140 (Texas Instruments Inc., Dallas, Tex., USA) which has a low voltage offset of 120 μN, an offset voltage drift of 1 μV/° C., a differential input impedance of 10¹³Ω, an output impedance of 16Ω and a gain bandwidth product of 11 MHz.

Surface Protection with Electropolymerised m-Phenylenediamine (mPD)

The final step when preparing the needle microelectrodes is to protect the working electrode from contamination and to only allow molecules on the scale of H₂O₂ to reach the surface. This technique has evolved from multiple reports of polymer films used to entrap enzymes by the electrode surface to form biosensors that exist in the literature, including films of nafion [64], polypyrrole [65], and polyphenol [66].

The most stable and uniform of these are formed by in-situ electropolymerisation. In this way the precise site, rate and thickness of the final film can be controlled. We have found that polymerising meta-phenylenediamine (mPD) [67] produces reproducible thin films that are closely adherent to the surface of the working electrode and sufficiently dense as to prevent larger interfering redox species from reaching the electrode surface, such as ferrocene or those commonly found in biological systems (ascorbate, urate or paracetamol (N-(4-hydroxyphenyDacetamide)) whilst still permitting H₂O₂ at a rate sufficient to give good response times (<1 sec).

The method is straightforward. The needle microelectrode is suspended within a 100 mM solution of mPD in 10 mM phosphate buffered saline at pH 7.4, and a voltage of +0.7V (vs. AgIAgC1) is applied to the working electrode for 20 minutes until the current diminishes to an asymptotically low level. The electrode is then held at 0V for a further 2-5 minutes before being allowed to air dry, followed by rinsing in dH₂O. The quality of the mPD layer is then checked with cyclic voltammetry, wherein a good result is considered to have reduced the magnitude of the signal peak by 95%, with equal oxidation and reduction profiles and no evidence of silver contamination.

### Example 3

### Optimising 3 Enzyme System

All experiments used the enzymes creatininase (CNH-311; EC 3.5.2.10; 259 U/mg), creatinase (CRH-221; EC 3.5.3.3; 9.18 U/mg), and sarcosine oxidase (SAO-351; EC 1.5.3.1; 13.3 U/mg), purchased from Sorachim (Sorachim SA., Lausanne, Switzerland) who supply enzymes from Toyobo (Toyobo Co., Ltd., Osaka, Japan).

This process of refinement took a number of months to complete, exploring the optimal range of enzyme mixtures, buffers and layout of the LabSmith microfluidic system to enable robust detection of creatinine at low concentration.

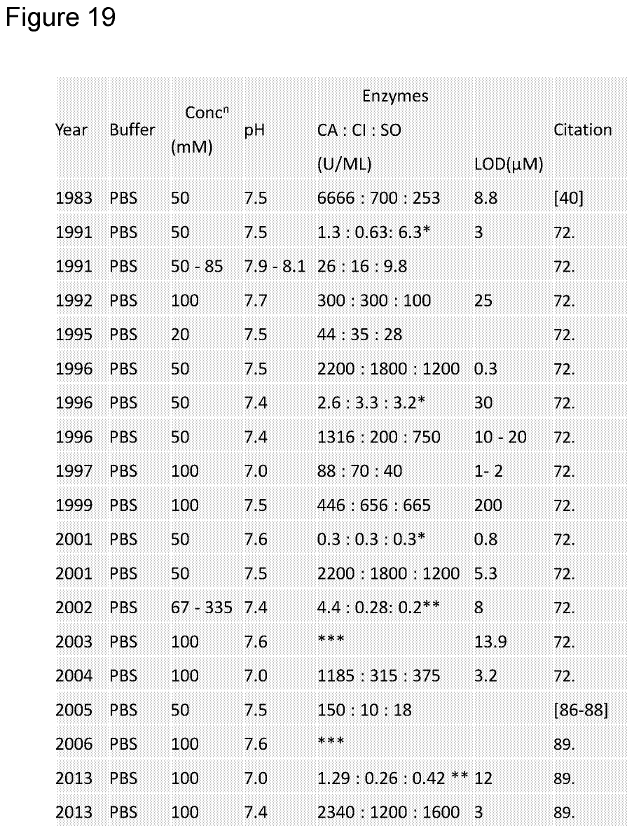

There were three noticeable trends after reviewing the literature regarding the selection and optimisation of the enzyme reaction. Firstly, the majority of researchers were using biosensors, with the enzymes embedded in a matrix applied directly to various forms of electrodes. Secondly, there was very little consistency in the specific amounts of enzyme used to create sensors nor the limits of detection derived therefrom. Thirdly, all research on this system over the past 33 years has used phosphate buffered saline (PBS) as the running buffer, see FIG. 19.

Of the papers presented in FIG. 19, only [73] and [74] did not use biosensors, employing instead spectrophotometric and flow-injection-analysis with a sequence of enzyme reaction beds, respectively.

### Example 4

### Buffer Selection

One reason for wishing to select a buffer other than PBS was the intended use of the system for sampling from either urine or blood. Table 1.1 shows that urinary pH can be as low as 4.5 (32 μmol of H⁺) in normal adults. I chose to over-design the system for a pH of 3, to maintain sensitivity in the face of severe ischaemia. The pKₐ of PBS is only 7.2, meaning that a highly concentrated buffer would be required to provide sufficient capacity to neutralise 1 mmol of H⁺ and maintain the pH of the dialysate within 0.1 unit of pH 8.0. This would require a PBS concentration of 100 mM, as demonstrated by using the Henderson-Hasselbalch equation as per 3.1 below, whereas a buffer with a pKₐ of 8.0 should only require a concentration of 20 mM to resist a pH change of ±0.1 unit.

$$8.0 = 7.2 + {log}_{10} ( \frac{Acid}{Base} ) \\ 1 {0}^{0.8} = ( \frac{Acid}{Base} )$$

$$Base ( 1 + 6.3095 ) = 100 mM$$

$$Base = 13.68 mM$$

$$Acid = 86.32 mM$$

Buffering 1 mmol of H+ would change the ratio as follows:

$$( \frac{86.32}{13.68} ) \to ( \frac{85.32}{14.68} )$$

Back-calculation with the Henderson-Hasselbalch Equation:

$$\begin{matrix}pH = 7.2 + {log}_{10} ( \frac{85.32}{14.68} ) \\ = 7.964\end{matrix}$$

I examined a range of alternate buffers, looking for a suitable buffer with a pKa of 8.0, low temperature susceptibility, and lack of cation complexation and identified 4-(2-Hydroxyethyl)piperazine-I-propanesulfonic acid (EPPS), an uncommon piperazine-based agent which matched all of these criteria.

Benchtop tests demonstrated that 50 mM of EPPS was able to neutralise a saline solution at pH 3.0 to a final pH of 7.7 when mixed in a 1:4 volumetric ratio with the buffered enzyme solution, versus just pH 7.5 for enzymes in 100 mM PBS.

### Example 5

### Optimisation Experiments

Previous work in the lab has found that a combination of perfusate flow at 2 μ1/min and enzyme at 0.5 μ1/min produce good results. I decided to work backwards from sarcosine oxidase to creatininase, directly testing and optimising each step in turn for the enzyme mixture and pH, prior to performing microdialysis experiments.

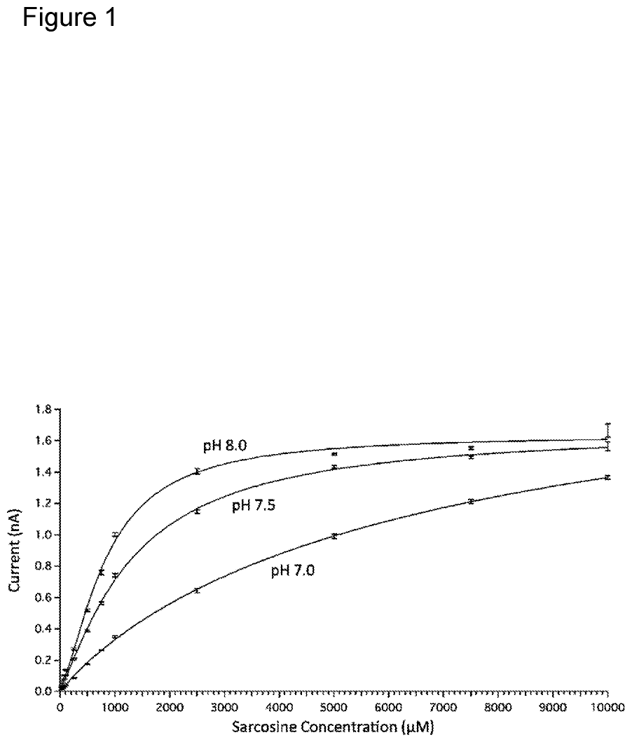

FIG. 1 shows the results of an initial set of experiments with a single 50 μm electrode which I ran prior to creating my 8×25 μm electrode, comparing the signal magnitude of 30 U/ml sarcosine oxidase in 10 mM PBS versus sarcosine from 25 μM to 10 mM, confirming my suspicions that basifying the pH to 8.0 would improve the signal. These results are similar for the two-step and three-step mixtures with higher sensitivity at pH 8.0 than 7.5.

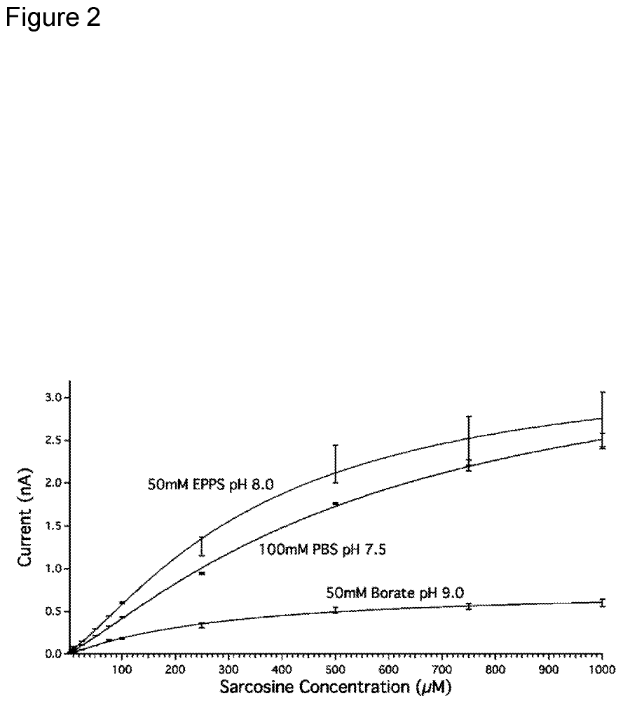

A head-to-head comparison of 30 U/ml SAO in 100 mM PBS at pH 7.5, 50 mM EPPS at pH 8.0 and 50 mM borate buffer at pH 9.0 provided the results in FIG. 2, using the newer 8×25 μm electrode array. The broadening of the standard deviation in the EPPS signal as the concentration progresses was most likely due to a fault with the substrate pump which also appeared in later experiments, leading to its replacement.

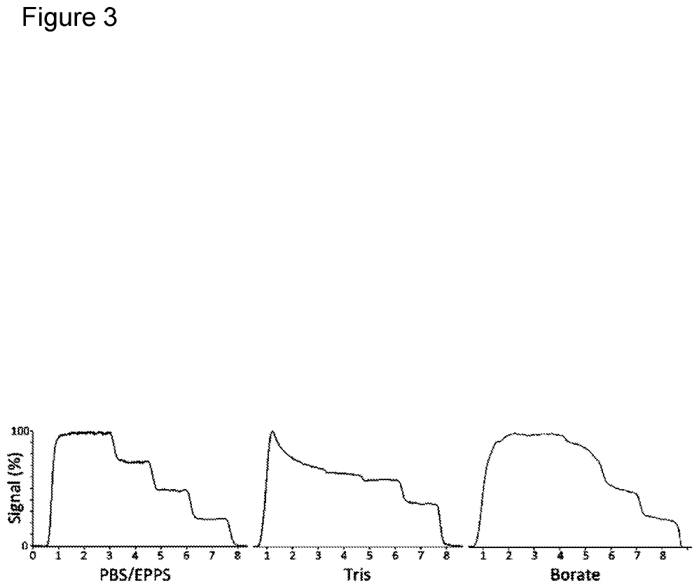

FIG. 3 shows the stepped profiles of these serial dilution experiments, demonstrating the clear results obtained in PBS and EPPS versus those from Tris and borate buffers, further confirming their unsuitability for this system.

Thereafter followed a series of experiments to examine the time profile of the response curves to various mixtures of enzymes to achieve the maximal response in the shortest time, beyond which minimal improvements could be seen. This would indicate that the enzyme ratios were no longer limiting, merely the amount of enzyme. I decided to limit the total enzyme content of the system (in weight/volume) to that of serum albumin (400 mg/ml), but this could be pushed further in later developments. I was mindful of the possibility of encrustation within the microfluidic system, as well as increased viscosity and interference with mixing and substrate diffusion at higher protein concentrations on these scales.

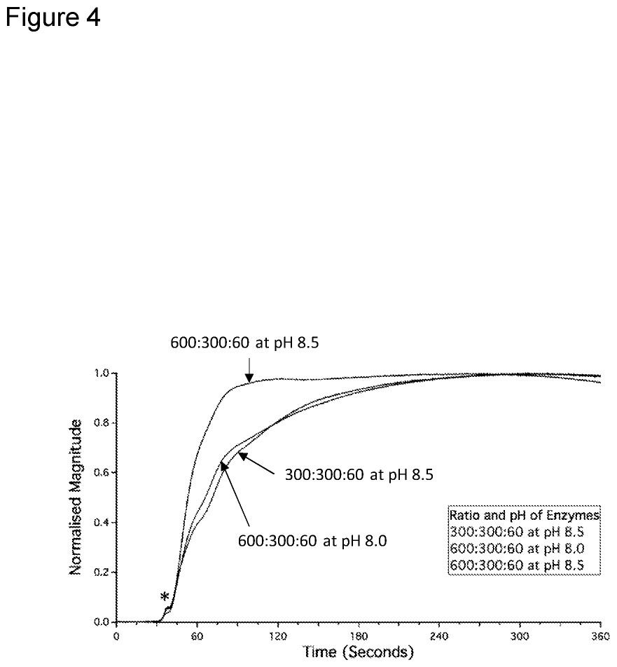

All experiments were carried out with a reservoir of 100 μM substrate in normal saline at pH 3.0 into which the enzyme mixture was added in the intended 1:4 volumetric ratio and then pumped past a sensor at 2.5 μl/minute to reproduce the total flow of the final system. The enzyme mixtures were buffered in 50 mM EPPS at pH 7.5, 8.0 and 8.5. The extensive series results will not be reproduced here, except for FIG. 4 which was one of the final experiments wherein the SAO and creatinase content had been optimised for 100 μm creatine, and this experiment was now attempting to ascertain the optimal amount of creatininase for 100 μm creatinine in normal saline at pH 3.0.

Note how increasing the pH from 8.0 to 8.5 was the equivalent of doubling the amount of creatininase content from 300 U/ml to 600 U/ml (blue vs. red lines), and the increased response with a mixture of 600:300:60 at pH 8.5. The final mixture chosen for the microdialysis experiments was 600:300:60 in 50 mM EPPS at pH 8.0, but this experiment raised the possibility of using an alternative buffering agent with a higher pKa around 8.5 in future, such as HEPBS (pKa of 8.3) [94].

Table 3.4 below presents a collection of T₉₀ levels (time to reach 90% of maximum, measured from the beginning of the upstroke) obtained by this experimental method at pH 8.0, demonstrating the evolution of the mixture.

TABLE 3.4

| SAO | T₉₀ | CRH:SAO | T₉₀ | CNH:CRH:SAO | T₉₀ |
|---|---|---|---|---|---|
| (U) | (sec) | (U) | (sec) | (U) | (sec) |
| 15 | 138 | 150:60 | 145 | 150:300:60 | 195 |
| 30 | 73 | 180:60 | 104 | 300:300:60 | 154 |
| 60 | 28 | 300:60 | 77 | 600:300:60 | 135 |

Results of enzyme optimisation experiments at pH 8.0 in order to achieve minimum T₉₀ levels. The reaction time of the final mixture is highlighted in bold.

From these results I decided to implement a 3 minute delay between the Y-junction feeding the enzyme into the dialysate, and the sensor, to ensure maximum sensitivity by providing adequate mixing and reaction time.

### Example 6

### Microdialysis Experiments

With the enzyme quantities and buffer optimised for detecting creatinine at levels of 100 μM, I moved to test the system in a simulated final setting with microdialysis. Here, a clinical-grade CMA 70 microdialysis probe (M Dialysis AB, Stockholm, Sweden) designed for deep tissue sampling, with a membrane surface area of 18.8 mm² and cut-off of 20 kDa was suspended in well-stirred T1 solution (an extracellular fluid analog) (our stock solution contains 2.3 mM calcium chloride, 147 mM sodium chloride, and 4 mM potassium chloride in dH₂O) to which was added aliquots of creatinine in a standard-addition methodology. T1 was also used as the perfusate, delivered at 2 μl/min by a Harvard Apparatus PHD 2000 programmable infusion pump (Harvard Bioscience Inc., Holliston, Mass., USA), with the dialysate returning into the Y-junction of my LabSmith board to mix with the buffered enzyme mixture flowing at 0.5 μl/min, followed by the delay loop and sensor. From these results it was possible to build a calibration curve for the system, which fit the Hill Equation for enzyme kinetics with a Km of 2.3 mM (±1.3 mM), Vₘₐₓ of 2.9 mM (±1.0 mM) and rate constant of 0.96 μM/sec (+0.05 μM/sec). Interestingly, the system's Km value encompasses that of sarcosine oxidase (Km of 2.8 mM), but not creatinine (4.5 mM) or creatininase (32 mM) which could indicate that this is the rate limiting step, perhaps even due to the availability of oxygen in solution (≠250 μm).

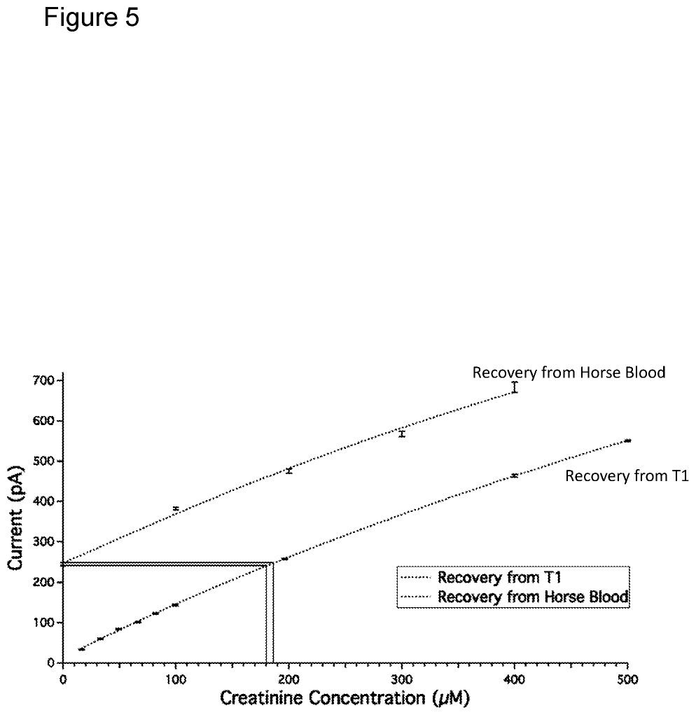

The same setup was then used for standard addition experiments in well-stirred defibrinated horse blood (TCS Biosciences Ltd., Botolph Claydon, Buckingham, UK) to prove that it was possible to detect micromolar quantities of creatinine in a biological fluid. The results are given in FIG. 5.

The results obtained in T1 show that this microdialysis setup, the first of its kind, is a sensitive and low-noise method for measuring creatinine, with a limit of detection of 4.3 μM and tested upper range of 500 μM. The Km of the curve indicates that this method could be useful up to levels around 2 mM after further testing, providing a o broad useful working range. Furthermore, the microdialysis sampling methodology only had an estimated recovery of 40%, meaning that improving recovery could push the limit of detection down to ≈2 μM.

The results in well-stirred horse blood show that the horse had a basal creatinine level of 180 μM-186 μM. This is just beyond the upper range of normal for a horse (100 μM-160 μM), but we do not know the muscle mass or gender of the horse from which this was obtained, nor their exercise status, whereby levels can rise to ≥200 μM [95]. It is also possible that the sample was slightly haemolysed, with erythrocyte creatine feeding into the enzyme cascade (see Table 3.5 below). The broader standard deviations of these results no doubt come from a combination of convection effects and excluded diffusion paths due to red cell mass, altering flux across the dialysis membrane in a chaotic fashion.

### Example 7

### Stability Testing

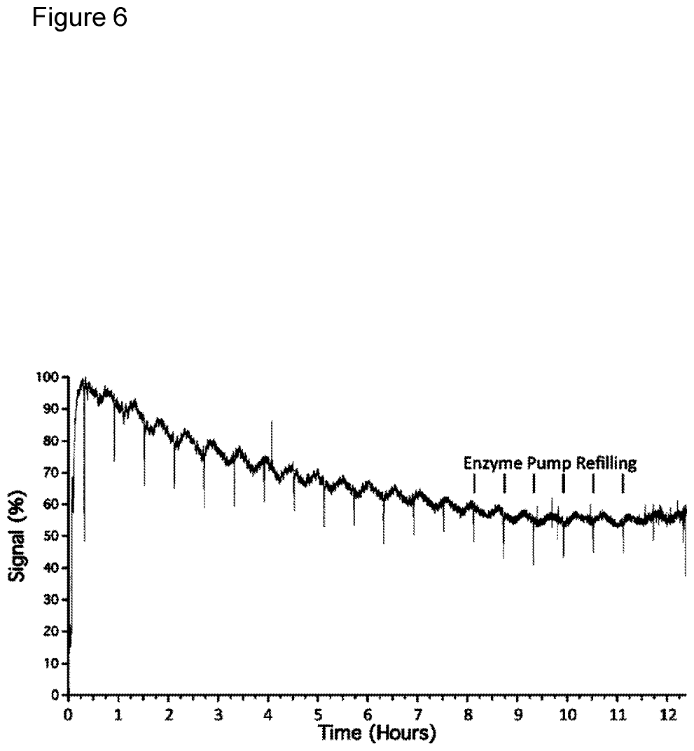

In order to test the long-term stability of this microdialysis system, I suspended the probe in a well-stirred pot of T1 to which was added an amount of creatinine to bring the total concentration to 100 μM. The normalised results in FIG. 6 show that the system remains responsive over a 12 hour period, with the sensitivity falling to a band between 50-60% of the original signal after 9 hours (equivalent to 250 pA), but remaining constant from that point onwards. The increase in noise from the 11% hour mark was the result of colleagues coming in to work in the morning. Spectral analysis showed three major noise peaks—one at 50 Hz from the power supply, a second one at 13 Hz possibly from the magnetic stirrer, and a much slower 0.2 Hz sinusoid superimposed visible over the entire dataset which could reflect convection within the stirred liquid or the screw drive of the Harvard Apparatus PHD 2000 pump.

### Example 8

### Interference Testing

At a bias voltage of 700 mV versus AgIAgC1, the working electrode is able to oxidise other chemicals often found in blood such as paracetamol, uric acid, and ascorbate, but these should be prevented from reaching the electrode surface by the polymerised mPD layer. The three-enzyme system will also be able to generate H₂O₂ from sarcosine and creatine.

The levels of these common interferents are presented below in Table 3.5.

TABLE 3.5

| Interferent | Concentration | Citation |
|---|---|---|
| Ascorbate | 164 μm † | [77] |
| | 228 μM † | [81] |
| Uric Acid | 916 μM † | [81] |
| | 42-744 μM * | [96] |
| Paracetamol | 164 μM † | [77] |
| | 264 μM † | [81] |
| Sarcosine | 0.6-2.76 μM * | [97] |
| Creatine | 25 μM * | [98] |
| | 38.2-68.7 μM * | [99] |

† Levels used in sensor testing.
* Documented normal range in serum

I did not test for creatine and sarcosine interference in the final system because the endogenous levels of sarcosine are in the low micromolar range, and those of creatine should only cause problems in the event of extensive haemolysis, as the majority is intracellular. These two substances could also be accounted for by pre-treatment, background subtraction, or a parallel sampling pathway with a different enzyme mixture.

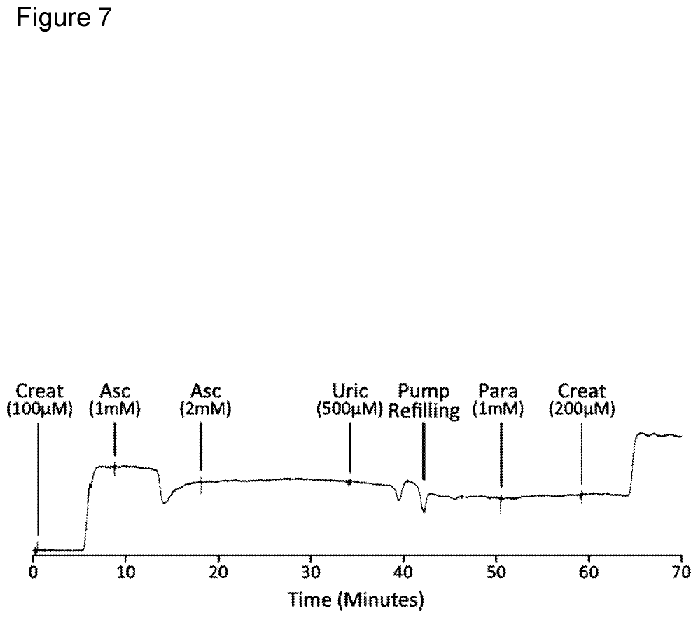

FIG. 7 presents the results of interference testing with the addition of the target substance into a well-stirred container of T1. The ascorbate and paracetamol were added in amounts exceeding those in the literature.

Note the response to the first addition of ascorbic acid but not the second, and a similar response to the addition of uric acid, prior to the pump refilling. These may have been due to a temporary reduction in recovery as the probe tip came into contact with the inside of the small glass sample pot used for the experiment. There is no apparent interference from the second addition of ascorbate, nor paracetamol, nor any interference with the response to a second aliquot of creatinine to bring the total concentration to 200 μM.

Despite these good results, I also realised that there could be a different way to discount the effects of any potential interferents in the system.

### Example 9

### Measuring Creatinine Clearance

Problems with measuring absolute magnitudes of responses include the need to continuously account for drift in the sensitivity and offset of the sensor as the working electrode becomes poisoned by H₂O₂, coated with protein, or the reference degrades. There is also a need to account for any potential interferents in the system which may give factitious results, as discussed in the previous section.

I realised that it should be possible to construct a test for the creatinine clearance itself, deriving the renal function as a rate constant rather than measuring absolute concentrations and thereby avoid all potential concerns over interferents and sensor drift, so long as the creatinine remains detectable above background. If we consider the closed loop perfusion system should contain no endogenous creatinine, it should be possible to add a known quantity of creatinine to the circulating volume at regular intervals and monitor for the decay rate as it is filtered into the urine by the working kidney with first-order kinetics. At levels above failure, the clearance should reflect the GFR, as the contribution by active tubular secretion is minimal.

I therefore constructed a series of experiments to simulate different creatinine clearance rates for known quantities of creatinine in T1 during continuous microdialysis sampling. For example, a clearance rate of 100 ml/min would bring a 1 litre sample circulating at a rate of 1/minute (equivalent to the blood circulation rate of a normal adult human (5 litres of blood at 51/min)) to half of its original concentration in five minutes. This clearance can be simulated by steadily doubling the volume of a 2 ml sample containing a known quantity of creatinine over five minutes, or at 400 μl/min. I chose to recreate the clearance rates of kidneys in various states of dysfunction, from CKD1 (Stage 1 Chronic Kidney Disease) to CKD4, with clearances of 100 ml/min, 75 ml/min, 50 ml/min and 25 ml/min respectively. Table 1.2 below first introduced the correspondence between the GFR and the stages of CKD. Note that the signal decay rate during stability testing as shown in FIG. 6 would be the equivalent to a clearance rate of 2 ml/min.

TABLE 1.2
The 5 stages of CKD. Stages 1 and 2 have preserved function
but with evidence of renal disease, such as scarring or the presence
of protein or blood in the urine. Stage 5 is also known as End-
Stage Renal Disease (ESRD), requiring dialysis or transplantation.

| Stage | 1 | 2 | 3 | 4 | 5 |
|---|---|---|---|---|---|
| GFR | >90 | 60-89 | 30-59 | 15-29 | <15 |
| (ml/min/1.73 m²) | | | | | |

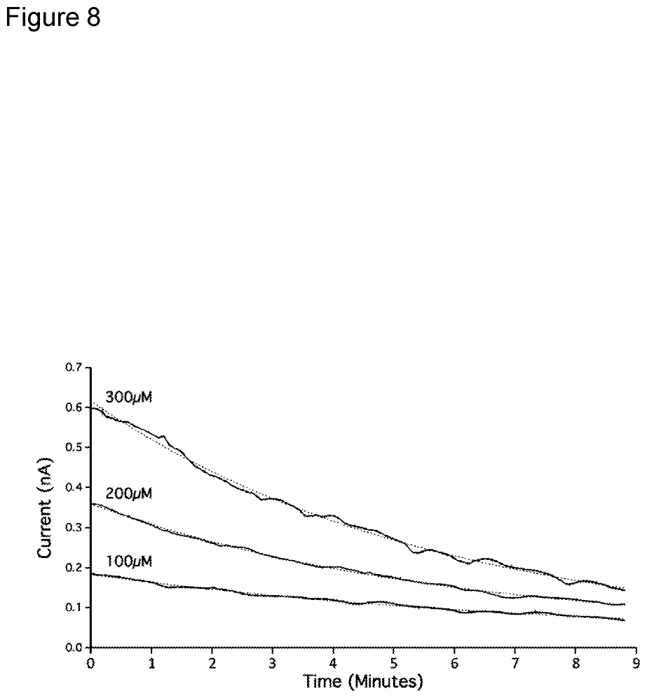

FIG. 8 shows the results of this dilution testing for three different concentrations of creatinine (100 μM, 200 μM and 300 μM) at a simulated clearance rate of 100 ml/min.

The results for the 200 μM and 300 μM experiments were very similar, with time constants giving half lives of 476±0.86 seconds and 471±1.0 seconds, respectively. The half life for the 100 μM sample was much higher at 620±2.8 seconds. It is worth noting that the decay curves are reminiscent of those described by the Albery equation [100], indicating the variability of the supply of substrate to the electrode in the dialysate is perhaps the root cause of these experimental errors, as I did not control for probe placement, stirring rate nor temperature.

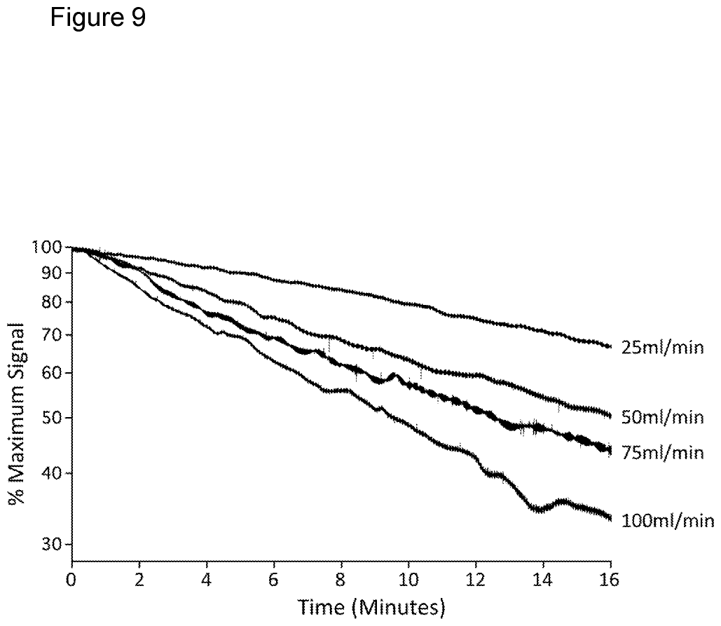

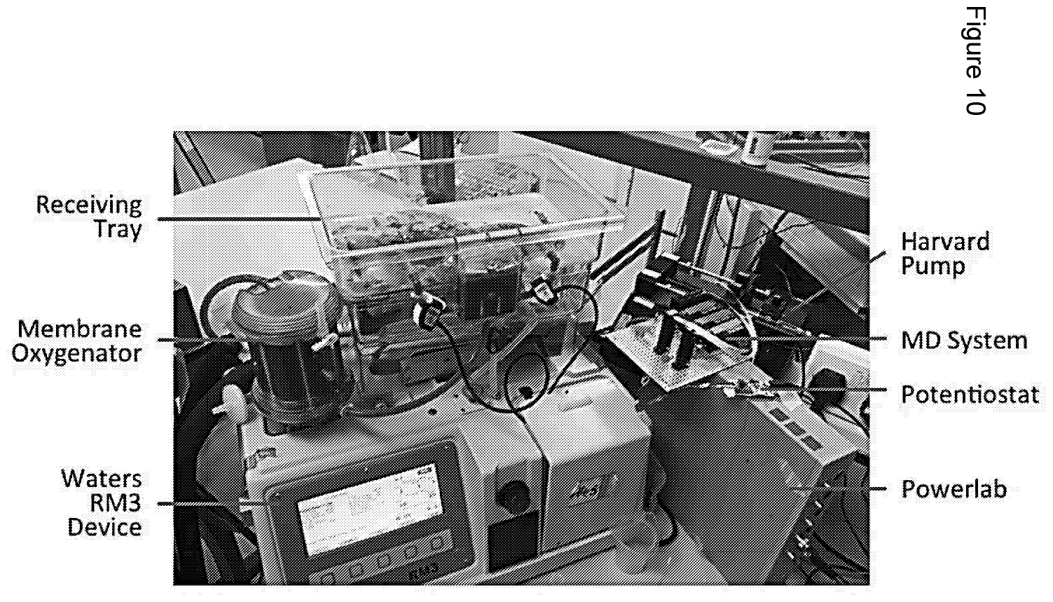

FIG. 9 shows the follow-up experiment to simulate different levels of CKD. Each signal has been standardised to begin at 100% to emphasise the different decay rates observed.

The half lives of these curves were derived from an exponential fit of the raw data, providing values around 13 mins 40 seconds, 16 mins 30 seconds, and 27 mins for the 75 ml/min, 50 ml/min and 25 ml/min clearance rates respectively. Whilst these do not directly correspond to the experimental design, they do follow an ordered sequence with some proportionality between the values obtained. The results are notably more stable at lower dilution rates, further implicating dialysis recovery and mixing as sources of error.

### Example 10

### Testing the System with a Blood-Perfused Porcine Kidney

The final experiment explored the function of this system in an isolated perfused kidney setup. To this end, I partnered with Dr. Bynvant Sandhu, a clinical researcher working at Hammersmith Hospital, one of the UK's major renal transplantation centres. Her work involved warm blood perfusion of porcine kidneys using an RM3 perfusion device (Waters Medical Systems LLC, Rochester, Minn., USA). An adult pig kidney was collected from a nearby licensed abattoir and maintained in static cold storage for 4 hours. Following this, it was connected into an RM3 perfusion device which had been reconfigured with a heat exchanger and oxygenator for warm perfusion. The autologous blood collected for the reperfusion experiment was visibly haemolysed and contained large amounts of thrombus which had to be filtered out prior to use.

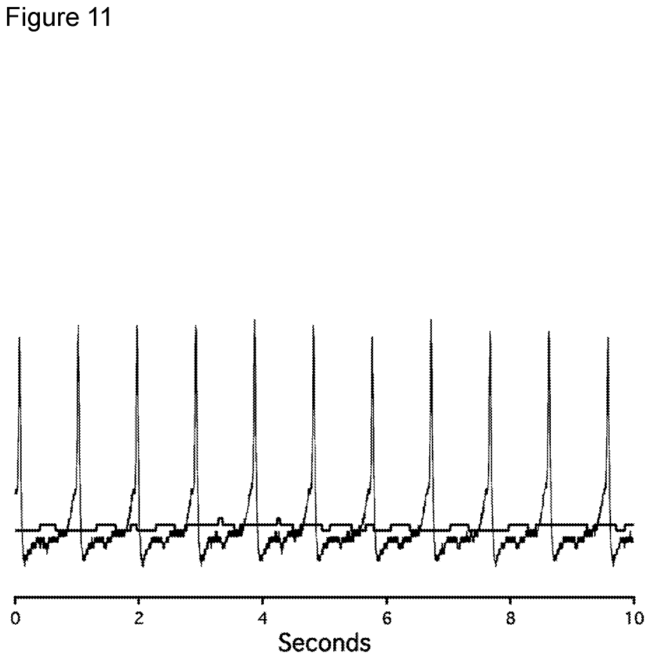

After calibrating the sensor system against 100 μM creatinine directly infused into the Y-junction and then via the microdialysis probe in an unstirred 100 μM creatinine-T1 solution, I placed the probe tip deep into the stump of the renal vein to ensure good flow. FIG. 10 shows the experimental setup in more detail. Data was then collected over the next hour of reperfusion until the probe membrane became damaged during repositioning and the experiment had to be abandoned.

Data analysis first required the use of a Savitsky-Golay smoothing filter (2nd order polynomial with a window of 513 samples) to remove the visible electrical spikes caused by the RM3's perfusion pump, as shown in FIG. 11.

These results show an initial plateau during system setup and initial perfusion, equivalent to ≈300 μM creatinine. This high system offset is probably due to a combination of the muscle damage from the slaughtering process, and extensive haemolysis of the autologous blood, releasing creatine into the perfusate. When perfusion first began, the blood noticeably darkened as the kidney began consuming oxygen. Opening up the oxygen supply to the membrane oxygenator quickly returned the blood to a ruby red colour and caused a sudden decrease in the signal magnitude which soon returned to the high baseline. This may have in fact reflected a sudden oxidative burst from the ischaemic kidney consuming the oxygen required for the sarcosine oxidase to function normally, or a rapid change in pH which was detected by the sensor.

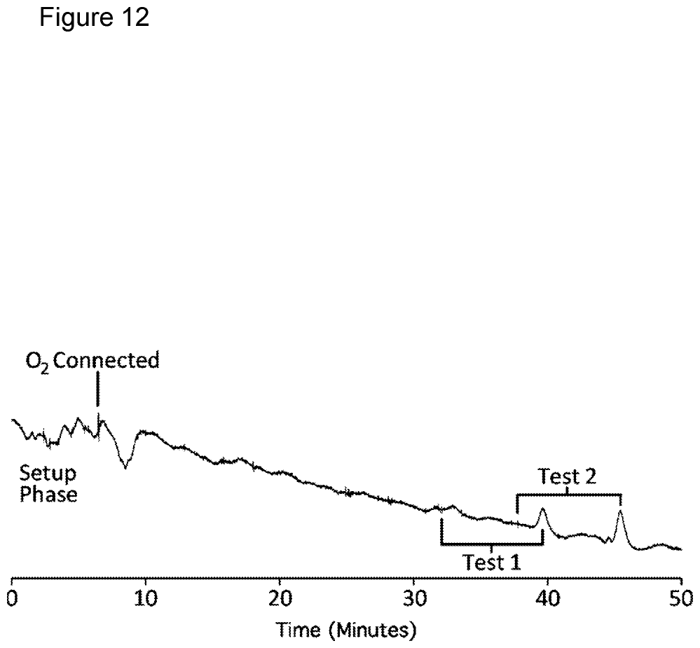

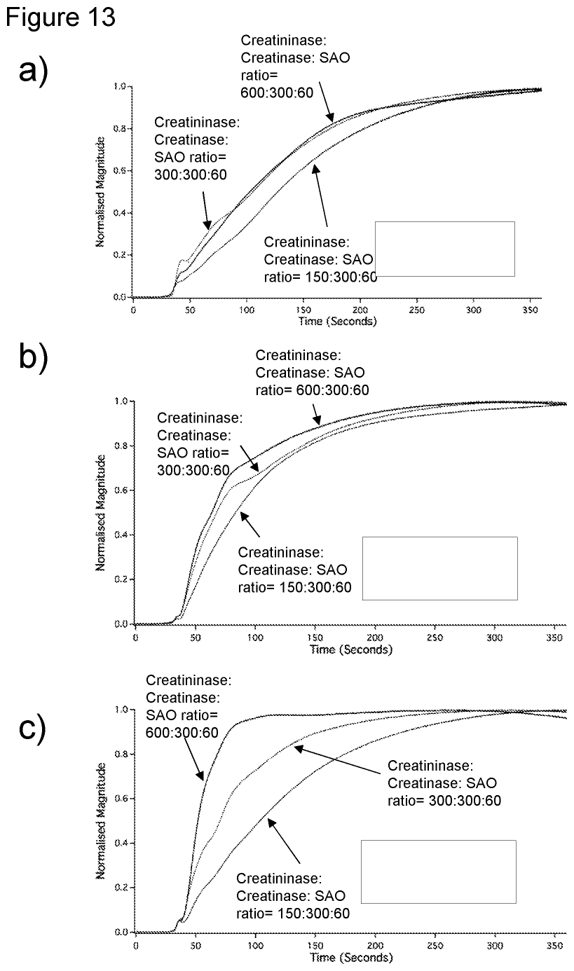

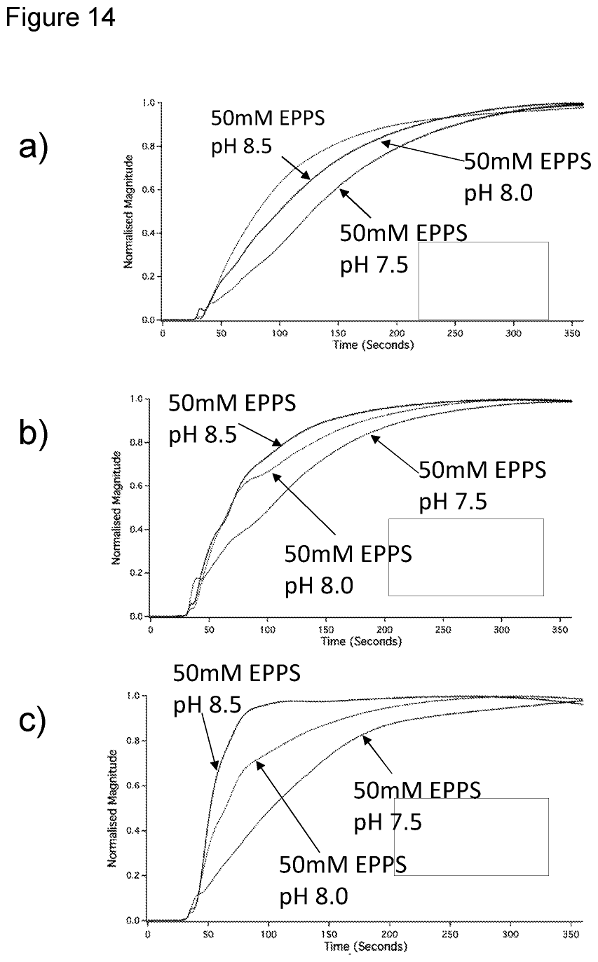

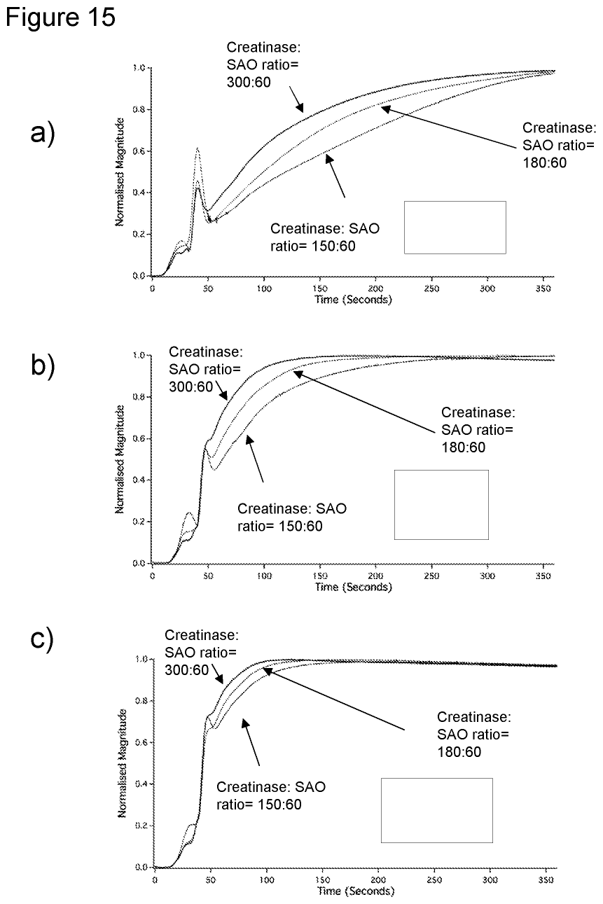

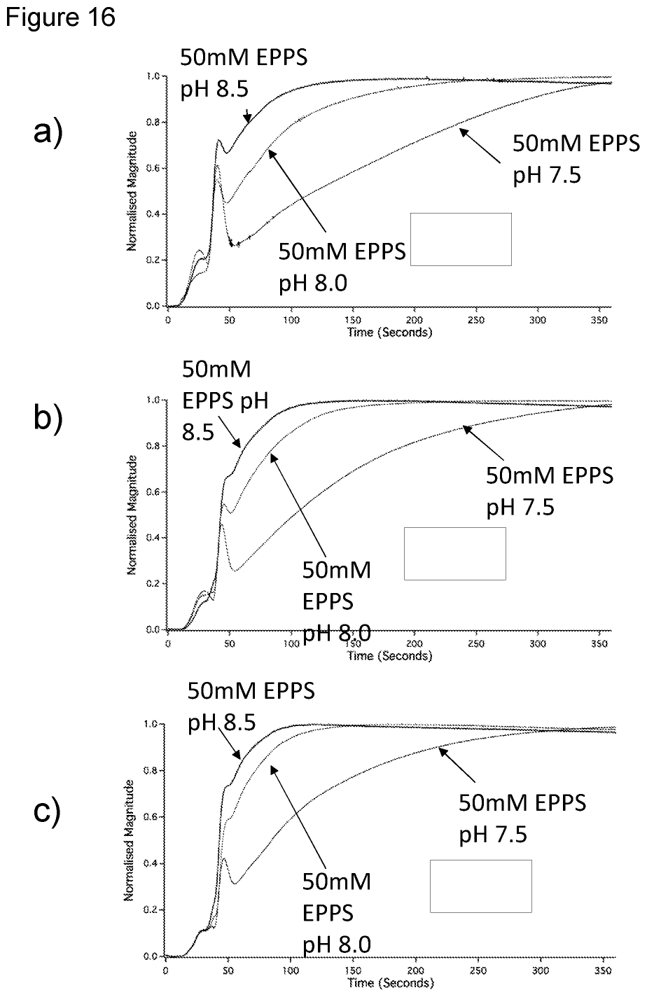

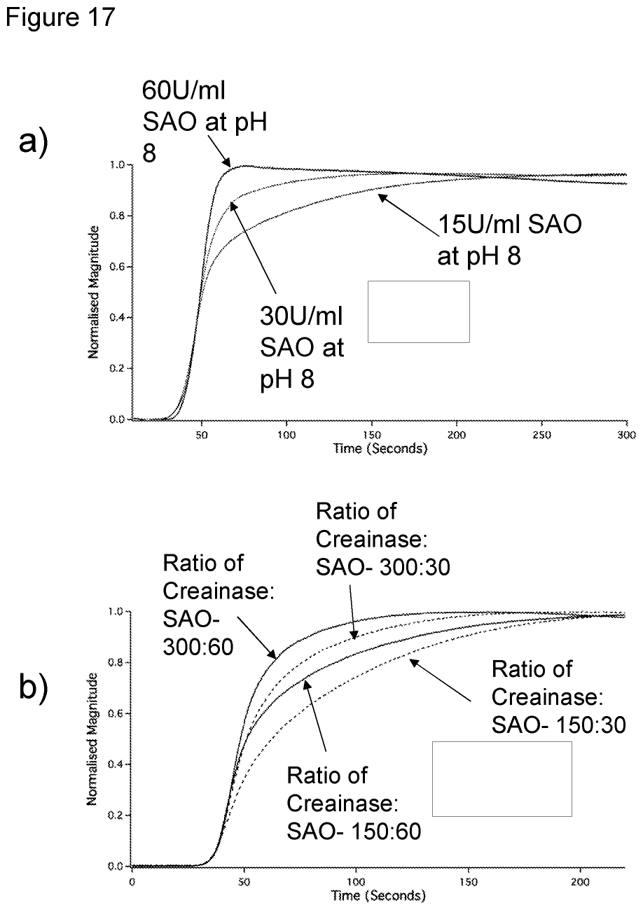

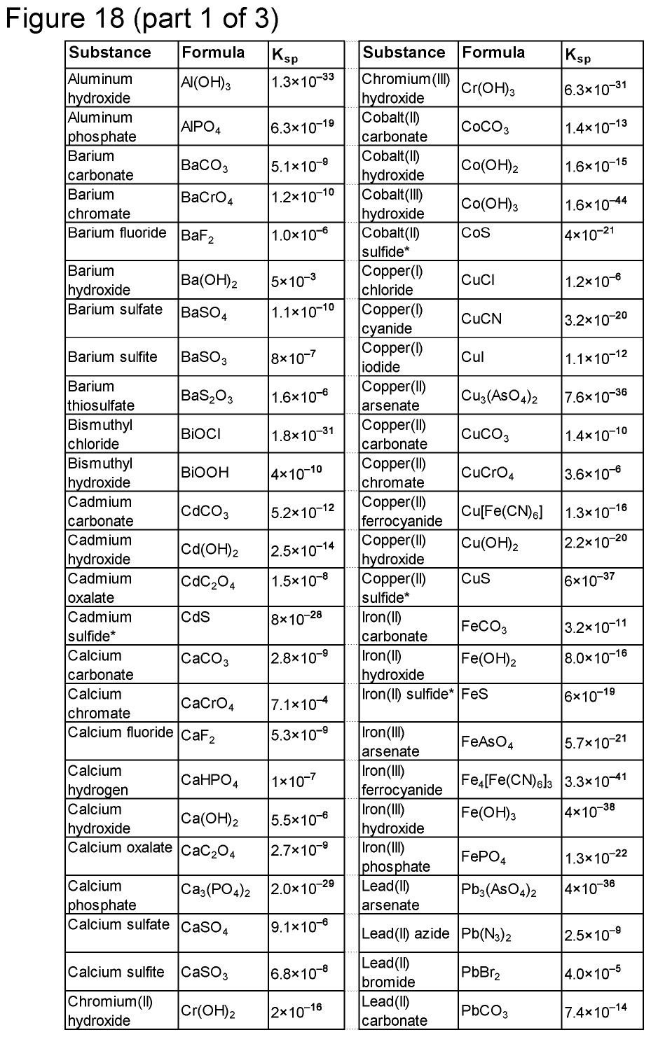

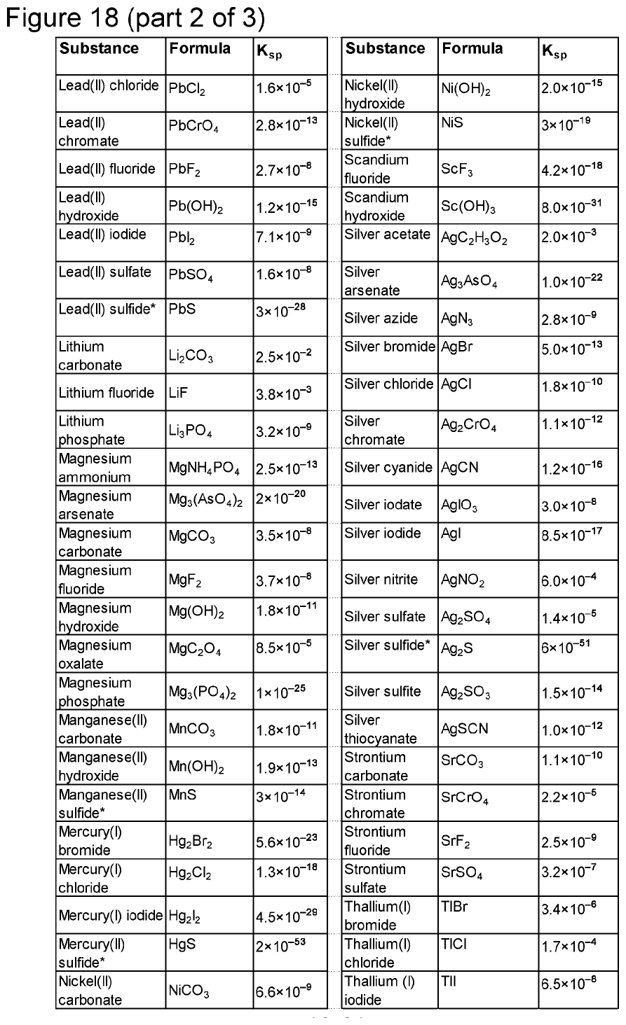

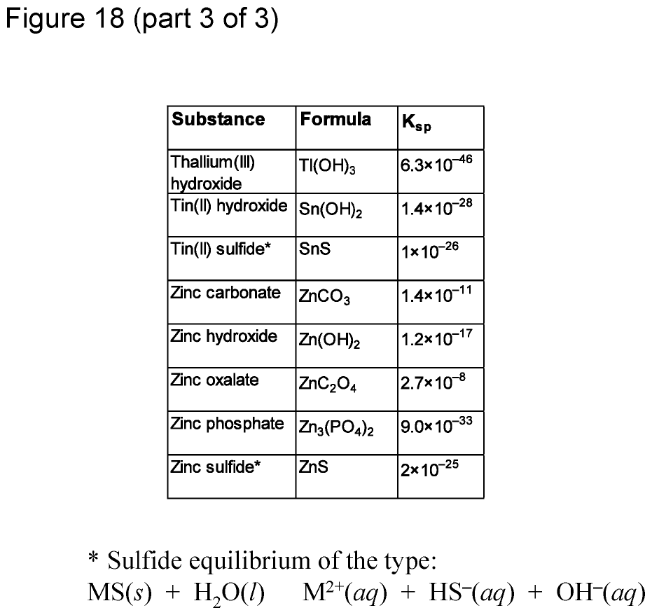

The kidney then appeared to be excreting detectable metabolites at a rate equivalent to that of the previous 100 ml/min creatinine clearance experiment, with a half-life of 652±3.5 seconds, with the caveat that the results may not be entirely equivalent. I then spiked the arterial reservoir of the RM3 system with two separate aliquots of 100 μmoles of creatinine (10 mls×10 mM), producing the results seen in FIG. 12. These curves had half-lives of 27 seconds and 18 seconds respectively, indicating that these results were more likely due to dilution than clearance.

Unfortunately the experiment had to be ended before the detectable metabolites in the system had been reduced to a low steady state. In the final reperfusion system as imagined, the perfusate would comprise washed erythrocytes in an isotonic crystalloid solution without any endogenous creatinine, thus allowing for pure clearance testing.

Conclusions

This part of the project has shown that a self-contained system based upon microdialysis sampling and amperometric testing of creatinine is able to achieve a limit of detection of 4.3 μM and tested upper range of 500 μM, matching or exceeding those reported in the literature (Table 3.3). This performance was due to a series of improvements and optimisations I made to the potentiostat, microelectrode sensor array and the triple-enzyme system of Tsuchida and Yoda [40]. The process of electropolymerising mPD onto the working electrode also provided good protection against levels of interferents far in excess of those reported by other groups performing such testing.

In addition to the development of a real-time creatinine monitoring system (with a 3 minute delay for reaction time) I have proposed and explored a novel way to monitor renal function without sensor calibration, thereby avoiding the need to compensate for any background noise or change in sensor offset, drift, or loss of sensitivity over time. I believe that this can be achieved by measuring the time constant (or half life) of the decay curve of creatinine excretion, and have demonstrated this experimentally in a closed-loop perfusion system containing a porcine kidney.

The economics of using this microfluidic system for real-time monitoring are also favourable. Despite the continuous wastage of the enzyme used in the analysis, a week of continuous monitoring would only consume 5 ml of the 600:300:60 mixture. At current market prices for the three enzymes as of September 2016, this would amount to less than £50/week.

Future work would see the enzymes re-optimised in a buffer with higher pKₐ such as HEPBS, the creation of a modular microdialysis sampling probe for in-line inclusion in a perfusion circuit, and an attempt to standardise the formation of a microelectrode array within a microchannel to provide a ‘hot-pluggable’ system for live creatinine monitoring. The system may also benefit from using droplet microfluidics to allow the multiplexing of multiple enzyme reactions in parallel with a common sensor whilst producing better mixing and less Taylor dispersion to reduce signal magnitude, as is probably occurring in the 3-minute delay loop. With further development, this system could also be trialled for monitoring live renal function in an intensive-care setting.

Overall, the present invention brings us closer to the goal of maintaining organs in optimal condition prior to transplantation, buying time in a setting where every second counts.

### REFERENCES

33. Randviir E P, Banks C E. Analytical methods for quantifying creatinine within biological media. Sensors and Actuators B: Chemical. 2013; 183:239-252.

35. Myers G L, Miller W G, Coresh J, Fleming J, Greenberg N, Greene T, et al. Recommendations for improving serum creatinine measurement: a report from the Laboratory Working Group of the National Kidney Disease Education Program. Clinical Chemistry. 2006; 52(1):5-18.

37. Panteghini M. Enzymatic assays for creatinine: time for action. Scandinavian Journal of Clinical and Laboratory Investigation. 2008; 68(sup241):84-88.

40. Tsuchida T, Yoda K. Multi-enzyme membrane electrodes for determination of creatinine and creatine in serum. Clinical Chemistry. 1983; 29(1):51-55.

51. Xu J C, Hunter G W, Lukco D, Liu C C, Ward B J. Novel carbon dioxide microsensor based on tin oxide nanomaterial doped with copper oxide. IEEE Sensors Journal. 2009; 9(3):235-236.

52. Suzuki H, Arakawa H, Karube I. Performance characteristics of a urea microsensor employing a micromachined carbon dioxide electrode. Electroanalysis. 2000; 12(16):1327-1333.

53. Halliwell B, Clement M V, Long L H. Hydrogen peroxide in the human body. FEBS letters. 2000; 486(1):10-13.

55. Ungerstedt U, Pycock C. Functional correlates of dopamine neurotransmission. Bulletin der Schweizerischen Akademie der Medizinischen Wissenschaften. 1974; 30(1-3):44-55.

56. Whitesides G M. The origins and the future of microfluidics. Nature. 2006; 442(7101):368-373.

58. Wang C. Development of clinical instruments for traumatic brain injury patients: design, realisation, and performance. Imperial College London; 2015.

64. Liu H, Ying T, Sun K, Li H, Qi D. Reagentless amperometric biosensors highly sensitive to hydrogen peroxide, glucose and lactose based on N-methyl phenazine methosulfate incorporated in a Nafion film as an electron transfer mediator between horseradish peroxidase and an electrode. Analytica Chimica Acta. 1997; 344(3):187-199.

65. Guerrieri A, De Benedetto G, Palmisano F, Zambonin P. Electrosynthesized non-conducting polymers as permselective membranes in amperometric enzyme electrodes: a glucose biosensor based on a co-crosslinked glucose oxidase/overoxidized polypyrrole bilayer. Biosensors and Bioelectronics. 1998; 13(1):103-112.

66. Nakabayashi Y, Wakuda M, Imai H. Amperometric Glucose Sensors Fabricated by Electrochemical Polymerization of Phenols on Carbon Paste Electrodes Containing Ferrocene as an Electron Transfer Mediator. Analytical Sciences. 1998; 14(6):1069-1076.

67. Mitchell K M. Acetylcholine and Choline Amperometric Enzyme Sensors Characterized in Vitro and in Vivo. Analytical Chemistry. 2004 February; 76(4):1098-1106. Available from: http://dx.doi.org/10.1021/ac034757v.

72. Khue N V, Wolff C M, Seris J L, Schwing J P. Immobilized enzyme electrode for creatinine determination in serum. Analytical Chemistry. 1991; 63(6):611-614.

73. Weber J, Van Zanten A. Interferences in current methods for measurements of creatinine. Clinical Chemistry. 1991; 37(5):695-700.

74. Sakslund H, Hammerich O. Effects of pH, temperature and reaction products on the performance of an immobilized creatininase-creatinase-sarcosine oxidase enzyme system for creatinine determination. Analytica Chimica Acta. 1992; 268(2):331-345.

75. Yamato H, Ohwa M, Wernet W. A polypyrrole/three-enzyme electrode for creatinine detection. Analytical Chemistry. 1995; 67(17):2776-2780.

76. Schneider J, Gründig B, Renneberg R, Cammann K, Madaras M, Buck R, et al. Hydrogel matrix for three enzyme entrapment in creatine/creatinine amperometric biosensing. Analytica Chimica Acta. 1996; 325(3): 161-167.

77. M\check{a}d\check{a}raç M B, Popescu I C, Ufer S, Buck R P. Microfabricated amperometric creatine and creatinine biosensors. Analytica Chimica Acta. 1996; 319(3):335-345.

78. M\check{a}d\check{a}raç M B, Buck R P. Miniaturized biosensors employing electropolymerized permselective films and their use for creatinine assays in human serum. Analytical Chemistry. 1996; 68(21):3832-3839.

79. Khan G, Wernet W. A highly sensitive amperometric creatinine sensor. Analytica Chimica Acta. 1997; 351(1):151-158.

80. Kim E J, Haruyama T, Yanagida Y, Kobatake E, Aizawa M. Disposable creatinine sensor based on thick-film hydrogen peroxide electrode system. Analytica Chimica Acta. 1999; 394(2):225-231.

81. Shin J H, Choi Y S, Lee H J, Choi S H, Ha J, Yoon I J, et al. A planar amperometric creatinine biosensor employing an insoluble oxidizing agent for removing redox-active interferences. Analytical Chemistry. 2001; 73(24):5965-5971.

82. Tombach B, Schneider J, Matzkies F, Schaefer R M, Chemnitius G C. Amperometric creatinine biosensor for hemodialysis patients. Clinica Chimica Acta. 2001; 312(1):129-134.

83. Erlenkötter A, Fobker M, Chemnitius G C. Biosensors and flow-through system for the determination of creatinine in hemodialysate. Analytical and Bioanalytical Chemistry. 2002; 372(2):284-292.

84. Stefan R I, Bokretsion R G, van Staden J F, Aboul-Enein H Y. Simultaneous determination of creatine and creatinine using amperometric biosensors. Talanta. 2003; 60(6):1223-1228.

85. Hsiue G H, Lu P L, Chen J C. Multienzyme-immobilized modified polypropylene membrane for an amperometric creatinine biosensor. Journal of Applied Polymer Science. 2004; 92(5):3126-3134.

86. Berberich J A, Yang L W, Madura J, Bahar I, Russell A J. A stable three-enzyme creatinine biosensor. 1. Impact of structure, function and environment on PEGylated and immobilized sarcosine oxidase. Acta Biomaterialia. 2005; 1(2):173-181.

87. Berberich J A, Yang L W, Bahar I, Russell A J. A stable three enzyme creatinine biosensor. 2. Analysis of the impact of silver ions on creatine amidinohydrolase. Acta Biomaterialia. 2005; 1(2):183-191.

88. Berberich J A, Chan A, Boden M, Russell A J. A stable three-enzyme creatinine biosensor. 3. Immobilization of creatinine amidohydrolase and sensor development. Acta Biomaterialia. 2005; 1(2): 193-199.

89. Staden RISv, Bokretsion R G. Simultaneous Determination of Creatine and Creatinine using Monocrystalline Diamond Paste-Based Amperometric Biosensors. Analytical Letters. 2006; 39(11):2227-2233.

90. Nieh C H, Tsujimura S, Shirai O, Kano K. Amperometric biosensor based on reductive H2O, detection using pentacyanoferrate-bound polymer for creatinine determination. Analytica Chimica Acta. 2013; 767:128-133.

91. Serafín V, Hernández P, Agüí L, Yáñez-Sedeño P, Pingarrón J. Electrochemical biosensor for creatinine based on the immobilization of creatininase, creatinase and sarcosine oxidase onto a ferrocene/horseradish peroxidase/gold nanoparticles/multi-walled carbon nanotubes/Teflon composite electrode. Electrochimica Acta. 2013; 97:175-183.

92. Yoshimoto T, Tanaka N, Kanada N, Inoue T, Nakajima Y, Haratake M, et al. Crystal structures of creatininase reveal the substrate binding site and provide an insight into the catalytic mechanism. Journal of Molecular Biology. 2004; 337(2):399-416.

93. Hall S B, Khudaish E A, Hart A L. Electrochemical oxidation of hydrogen peroxide at platinum electrodes. Part IV: phosphate buffer dependence. Electrochimica Acta. 1999; 44(25):4573-4582.

94. Ferreira C M, Pinto I S, Soares E V, Soares H M. (Un)suitability of the use of pH buffers in biological, biochemical and environmental studies and their interaction with metal ions—a review. RSC Advances. 2015; 5(39):30989-31003.

95. Judson G, Frauenfelder H, Mooney G. Biochemical Changes in Thoroughbred Racehorses Following Submaximal and Maximal Exercise. In: Equine Exercise Physiology. Granta Editions Cambridge; 1983. p. 408-415.

96. Fang J, Alderman M H. Serum uric acid and cardiovascular mortality: the NHANES I epidemiologic follow-up study, 1971-1992. JAMA. 2000; 283(18):2404-2410.

97. Allen R H, Stabler S P, Lindenbaum J. Serum betaine, N,N-dimethylglycine and N-methylglycine levels in patients with cobalamin and folate deficiency and related inborn errors of metabolism. Metabolism. 1993 November; 42(11):1448-1460. Available from: http://dx.doi.org/10.1016/0026-0495(93)90198-W.

98. Schedel J M, Tanaka H, Kiyonaga A, Shindo M, Schutz Y. Acute creatine ingestion in human: consequences on serum creatine and creatinine concentrations. Life Sciences. 1999; 65(23):24632470.

99. Verhelst J, Berwaerts J, Marescau B, Abs R, Neels H, Mahler C, et al. Serum creatine, creatinine, and other guanidino compounds in patients with thyroid dysfunction. Metabolism. 1997; 46(9):1063-1067.

100. Albery W J, Haggett B G, Svanberg L R. The development of electrochemical sensors. In: Advanced Agricultural Instrumentation. Springer; 1986. p. 349-392.

The invention also provides the following numbered embodiments:

1. A composition comprising any two of or all of the enzymes creatininase, creatinase and sarcosine oxidase.

2. The composition of embodiment 1 wherein at least one, optionally two, optionally all of the enzymes are not immobilised, optionally wherein all of the enzymes are in solution.

3. The composition of embodiment 1 wherein the composition comprises a buffer.

4. The composition of embodiment 3 wherein the buffer is not a phosphate buffer or PBS, and/or is not a Tris buffer, and/or is not tetraborate and/or is not HEPES.

5. The composition of any one of embodiments 3 or 4 wherein the buffer is selected from the group consisting of EPPS, HEPBS, POPSO, HEPPSO and MOBS.

6. The composition of any one of embodiments 3-5 wherein the buffer has a pKa of between 7.0-9.0, optionally between 7.3-8.95, optionally 8.5.

7. The composition according to any one of embodiments 1-6 wherein the composition or the buffer is at a pH of between 7.0-9.0, optionally between 7.3-8.95, optionally 8.5.

8. The composition according to any one of embodiments 1-7 wherein the composition comprises EPPS at pH 8.0-8.5, optionally 50 mM EPPS at pH 8.0-8.5, optionally 50 mM EPPS at pH 8.0 or 50 mM EPPS at pH 8.5.

9. The composition of any one of embodiments 1-8 further comprising urease and/or uricase and/or means to detect Cystatin C and/or means to detect albumin.

10. The composition of any of the preceding embodiments wherein the creatininase is from Sorachim catalogue number CNH-311; and/or the creatinase is from Sorachim catalogue number CRH-211; and/or the sarcosine oxidase is from Sorachim catalogue number SAO-351.

11. The composition of any of the preceding embodiments wherein the concentration of creatininase and/or creatinase and/or sarcosine oxidase is such that in the final reaction mix the concentration of creatininase is at least 300 U/ml, and/or the concentration of creatinase is at least 120 U/ml and the concentration of sarcosine oxidase is at least 10 U/ml.

12. The composition of any of the preceding embodiments wherein the composition is such that the final mixed solution that results from the mixing of a sample which contains creatinine and the composition of any of the preceding embodiments comprises creatininase, creatinase, and sarcosine oxidase at a ratio of between 10:5:1 and 49:8:1 U/ml.

13. The composition of any of the preceding embodiments wherein the composition is such that the final mixed solution that results from the mixing of a sample which contains creatinine and the composition of any of the preceding embodiments comprises creatininase, creatinase, and sarcosine oxidase in the amounts of 600 U/ml, 300 U/ml and 60 U/ml, optionally wherein the composition is at pH 8.5.

14. A sensor system comprising creatininase and/or creatinase and/or sarcosine oxidase and at least a first sensor, optionally an amperometric sensor, optionally wherein the creatininase and/or creatinase and/or sarcosine oxidase are part of a composition according to any one of the preceding embodiments.

15. The sensor system according to embodiment 14 comprising any one of more of a microfluidic circuit, a microfluidic device, and a microdialysis probe.

16. The sensor system according to any one of embodiments 14 and 15 further comprising a continuous flow system.

17. The sensor system according to any of embodiments 14-16 wherein the system further comprises means to take a sample, optionally a sample from a patient or a sample from a closed-loop isolated perfused organ, optionally a kidney,
optionally wherein the sample from a patient is a microdialysate, optionally from blood, urine, plasma, tissue fluid, cerebrospinal fluid.

18. The sensor system according to any of the preceding embodiments arranged such that the creatininase and/or creatinase and/or sarcosine oxidase or the composition according to any one of the preceding embodiments is added to a sample prior to contacting the sample with the sensor, optionally wherein the sensing reagent is added more than 1, 2, 5, 10, 20, 30, 40, 50, 60, 70, 80, 90, 100, 110, 120, 130, 140, 150, 160, 170, 180, 190, 200, 225, 250 seconds, 5, 5.5, 6, 6.5, 7.5, 8, 8.5, 9, 9.5 or 10 minutes prior to contact with the sensor.

19. The sensor system of any of the preceding embodiments wherein the system comprises means to increase the amount of oxygen in the sample, either prior to or post addition of the sensing reagent, optionally wherein the means to increase the amount of oxygen are selected from any one or more of a:

a mixer, optionally that includes baffles or serpentine zones, optionally wherein the mixer is made out of a highly permeable material such as PDMS;

multiple mixing stages connected by Teflon tubing;

a pressurised container.

20. The sensor system of any of the preceding embodiments wherein the system can detect creatinine at a concentration of less than 10 uM, optionally less than 7.5 uM, optionally less than 5 uM, optionally less than 4 uM, optionally less than 3 uM, optionally less than 2 uM, optionally less than 1 uM.

21. The sensor system according to any of the preceding embodiments wherein the sensor system can detect a change in creatinine concentration of less than 1 uM, or less than 2 uM or less than 3 uM or less than 4 uM, or less than 5 uM or less than 7.5 uM or less than 10 uM, against a background level of creatinine of between 40 uM to 120 uM.

22. The sensor system of any of the preceding embodiments wherein the system comprises means for collecting data from the sensor, optionally a PowerLab/4SP, optionally wherein the system further comprises a wireless transmitting means for transmitting the data.

23. The sensor system of any of the preceding embodiments wherein the system further comprises means for data analysis, optionally a computer or wearable device, optionally wherein the means for data analysis comprise means for receiving wirelessly transmitted data.

24. The sensor system of any of the preceding embodiments further comprising at least one waste collection receptacle, optionally wherein the volume of the waste collection receptacle is less than 10 ml, for instance less than 9.5 ml, for instance less than 9 ml, for instance less than 8.5 ml, for instance less than 8 ml, for instance less than 7.5 ml, for instance less than 7 ml, for instance less than 6.5 ml, for instance less than 6 ml, for instance less than 5.5 ml, for instance less than 5 ml, for instance less than 4.5 ml, for instance less than 4 ml, for instance less than 3.5 ml, for instance less than 3 ml, for instance less than 2.5 ml, for instance less than 2 ml, for instance less than 1.5 ml, for instance less than 1 ml, for instance less than 0.5 ml, for instance less than 0.25 ml.

25. The sensor system of any of the preceding embodiments wherein the system is an ambulatory system.

26. The sensor system of any of the preceding embodiments wherein the system comprises the means to calculate the creatinine level/creatinine clearance rate/glomerular filtration rate.

27. The sensor system according to any of the preceding embodiments further comprising means to deliver an agent, optionally a contrast agent or a drug or creatinine, or creatine, or sarcosine, optionally wherein the means is a drug pump,
optionally wherein the drug is selected from the group consisting of immunosuppressants; chemotherapy agents such as platinum agents; antimicrobials such as the glycopeptides vancomycin and teicoplanin, and penicillin; and opioid analgesics such as morphine, diamorphine and codeine;
optionally wherein the amount of agent delivered is adjusted based on the calculated creatinine level/creatinine clearance rate/glomerular filtration rate.

28. The sensor system according to any of the preceding embodiments wherein the system further comprises a second sensor and optionally a second means to obtain a second sample, wherein the second sample is contacted with a second sensing reagent that comprises creatinase and sarcosine oxidase prior to detection at the second sensor, optionally wherein the system is arranged such that the second sensing reagent is added the to the second sample added more than 1, 2, 5, 10, 20, 30, 40, 50, 60, 70, 80, 90, 100, 110, 120, 130, 140, 150, 160, 170, 180, 190, 200, 225, 250 seconds, 5, 5.5, 6, 6.5, 7.5, 8, 8.5, 9, 9.5 or 10 minutes prior to contact with the sensor.

29. The sensor system according to embodiment 28 wherein the system comprises means to subtract the data obtained from the second sensor from the data obtained from the first sensor.

30. The sensor system according to any of the preceding embodiments wherein the first sensor captures data continuously.

31. The sensor system according to any of the preceding embodiments wherein the first sensor captures data at least every 24 hours, or at least every 22 hours, for example at least every 20 hours, for example at least every 18 hours, for example at least every 16 hours, for example at least every 14 hours, for example at least every 12 hours, for example at least every 10 hours, for example at least every 8 hours, for example at least every 6 hours, for example at least every 5 hours, for example at least every 4 hours, for example at least every 3 hours, for example at least every 2 hours for example at least every 1.5 hours, for example at least every 1 hour, for example at least every 50 minutes, for example at least every 45 minutes, for example at least every 40 minutes, for example at least every 35 minutes, for example at least every 30 minutes, for example at least very 25 minutes, for example at least every 20 minutes, for example at least every 15 minutes, for example at least every 10 minutes, for example at least every 5 minutes, for example at least every 2 minutes, for example at least every 1.5 minutes, for example at least every 60 seconds, for example at least every 45 seconds, for example at least every 30 seconds, for example at least every 15 seconds, for example at least every 10 seconds, for example at least every 5 seconds, for example at least every 2 seconds, for example at least every 1 second for example at least every 0.5 seconds.

32. A method for the determination of the level of creatinine in a sample from a human or animal subject, wherein the method comprises the use of the composition or sensor system according to any of the preceding embodiments, optionally wherein the sample is a dialysate or a microdialysate.

33. A method for the determination of the creatinine level and/or the creatinine clearance rate and/or the glomerular filtration rate wherein the method comprises the use of the composition or sensor system according to any of the preceding embodiments, optionally wherein the sample is a dialysate or a microdialysate.

34. A method for the real-time determination of the level of the creatinine level and/or the creatinine clearance rate and/or the glomerular filtration rate in a sample from a human or animal subject, wherein the method comprises the use of the composition of sensor system according to any of the preceding embodiments, optionally wherein the sample is a dialysate or a microdialysate.

35. A method for diagnosing a subject as having acute or chronic kidney disease, the method comprising determining the creatinine level and/or the creatinine clearance rate and/or the glomerular filtration rate according to any of the preceding methods, optionally further comprising treating the subject for acute or chronic kidney disease or stopping treatment with a drug that is contraindicated or dangerous in acute or chronic kidney disease, optionally wherein the drug is selected from the group consisting of
immunosuppressants; chemotherapy agents such as platinum agents; antimicrobials such as the glycopeptides vancomycin and teicoplanin, and penicillin; and opioid analgesics such as morphine, diamorphine and codeine.

36. The method of any of the preceding embodiments wherein determination of the level of the creatinine level and/or the creatinine clearance rate and/or the glomerular filtration rate is determined following administration of an amount of creatinine and/or creatine and/or sarcosine, optionally prior to and following administration of a drug.

37. The method of any of the preceding embodiments wherein the method further comprises administration of a dosage of a drug, wherein the dosage has been determined based on the creatinine level and/or the creatinine clearance rate and/or the glomerular filtration rate determined by the sensor system.

38. A method for monitoring a kidney for transplant, said method comprising perfusing the kidney and administering an amount of creatinine and/or creatine and/or sarcosine into the system, and determining the creatinine clearance rate using the composition and/or system and/or methods of any of the preceding embodiments.

39. A method for monitoring kidney function in a recipient of a transplant wherein the creatinine level and/or the creatinine clearance rate and/or the glomerular filtration rate is determined by use of the composition, sensor system and/or methods of any of the preceding embodiments.

40. A kit comprising:
any two or all of creatininase, creatinase and sarcosine oxidase; and/or
a composition according to any of the preceding embodiments;
creatinine and/or creatine and/or sarcosine; and/or
at least one waste receptacle;
a buffer, optionally a buffer according to any of the preceding embodiments;
a microdialysis probe; and/or
at least one, optionally at least two precision pumps.

1. A sensor system comprising sarcosine oxidase and/or creatininase and/or creatinase and at least a first sensor, optionally an amperometric sensor, optionally wherein the sarcosine oxidase and/or creatininase and/or creatinase are part of a composition.

2. The sensor system according to claim 1 wherein the composition comprises any two of or all of the enzymes sarcosine oxidase, creatininase and creatinase.

3. The sensor system according to any of claim 1 or 2 comprising sarcosine oxidase, creatininase and creatinase.

4. The sensor system according to claim 2 or 3 wherein at least one, optionally two, optionally all of the enzymes are not immobilised, optionally wherein all of the enzymes are in solution.

5. The sensor system according to claim 4 wherein the sarcosine oxidase, creatininase and creatinase are in solution.

6. The sensor system according to any of claims 1 to 5 further comprising a buffer, optionally wherein the composition comprises a buffer.

7. The sensor system according to claim 6 wherein the buffer is not a phosphate buffer or PBS, and/or is not a Tris buffer, and/or is not tetraborate and/or is not HEPES.

8. The sensor system according to any of claim 6 or 7 wherein the buffer is selected from the group consisting of EPPS, HEPBS, POPSO, HEPPSO and MOBS.

9. The sensor system according to any of claims 1-8 wherein the composition or the buffer is at a pH of between 7.0-9.0, optionally between 7.3-8.95, optionally 8.5.

10. The sensor system according to any of claims 1-9 wherein the composition comprises EPPS at pH 8.0-8.5, optionally 50 mM EPPS at pH 8.0-8.5, optionally 50 mM EPPS at pH 8.0 or 50 mM EPPS at pH 8.5.

11. The sensor system according to any of claims 1-10 wherein the composition further comprises urease and/or uricase and/or means to detect Cystatin C and/or means to detect albumin.

12. The sensor system according to any of claims 1-11 wherein the creatininase is from Sorachim catalogue number CNH-311; and/or the creatinase is from Sorachim catalogue number CRH-211; and/or the sarcosine oxidase is from Sorachim catalogue number SAO-351.

13. The sensor system according to any of claims 1-12 wherein the concentration of sarcosine oxidase and/or creatininase and/or creatinase in the composition is such that in the final reaction mix the concentration of creatininase is at least 300 U/ml, and/or the concentration of creatinase is at least 120 U/ml and the concentration of sarcosine oxidase is at least 10 U/ml.

14. The sensor system according to any of claims 1-13 wherein the composition is such that the final mixed solution that results from the mixing of a sample which contains creatinine and the composition of any of the preceding claims comprises creatininase, creatinase, and sarcosine oxidase at a ratio of between 10:5:1 and 49:8:1 U/ml.

15. The sensor system according to any of claims 1-13 wherein the composition is such that the final mixed solution that results from the mixing of a sample which contains creatinine and the composition of any of the preceding claims comprises creatininase, creatinase, and sarcosine oxidase in the amounts of 600 U/ml, 300 U/ml and 60 U/ml, optionally wherein the composition is at pH 8.5.

16. The sensor system according to any of claims 1-15 comprising any one of more of a microfluidic circuit, a microfluidic device, and a microdialysis probe.

17. The sensor system according to any one of claims 1-16 further comprising a continuous flow system.

18. The sensor system according to any of claims 1-17 wherein the system further comprises means to take a sample, optionally a sample from a patient or a sample from a closed-loop isolated perfused organ, optionally a kidney, optionally wherein the sample from a patient is a microdialysate, optionally from blood, urine, plasma, tissue fluid, cerebrospinal fluid.

19. The sensor system according to any of the preceding claims arranged such that the sarcosine oxidase and/or creatininase and/or creatinase or the composition according to any one of the preceding claims is added to a sample prior to contacting the sample with the sensor, optionally wherein the sensing reagent is added more than 1, 2, 5, 10, 20, 30, 40, 50, 60, 70, 80, 90, 100, 110, 120, 130, 140, 150, 160, 170, 180, 190, 200, 225, 250 seconds, 5, 5.5, 6, 6.5, 7.5, 8, 8.5, 9, 9.5 or 10 minutes prior to contact with the sensor.

20. The sensor system of any of the preceding claims wherein the system comprises means to increase the amount of oxygen in the sample, either prior to or post addition of the sensing reagent, optionally wherein the means to increase the amount of oxygen are selected from any one or more of a:
a mixer, optionally that includes baffles or serpentine zones, optionally wherein the mixer is made out of a highly permeable material such as PDMS;
multiple mixing stages connected by Teflon tubing;
a pressurised container.

21. The sensor system of any of the preceding claims wherein the system can detect creatinine at a concentration of less than 10 uM, optionally less than 7.5 uM, optionally less than 5 uM, optionally less than 4 uM, optionally less than 3 uM, optionally less than 2 uM, optionally less than 1 uM.

22. The sensor system according to any of the preceding claims wherein the sensor system can detect a change in creatinine concentration of less than 1 uM, or less than 2 uM or less than 3 uM or less than 4 uM, or less than 5 uM or less than 7.5 uM or less than 10 uM, against a background level of creatinine of between 40 uM to 120 uM.

23. The sensor system of any of the preceding claims wherein the system comprises means for collecting data from the sensor, optionally a PowerLab/4SP, optionally wherein the system further comprises a wireless transmitting means for transmitting the data.

24. The sensor system of any of the preceding claims wherein the system further comprises means for data analysis, optionally a computer or wearable device, optionally wherein the means for data analysis comprise means for receiving wirelessly transmitted data.

25. The sensor system of any of the preceding claims further comprising at least one waste collection receptacle, optionally wherein the volume of the waste collection receptacle is less than 10 ml, for instance less than 9.5 ml, for instance less than 9 ml, for instance less than 8.5 ml, for instance less than 8 ml, for instance less than 7.5 ml, for instance less than 7 ml, for instance less than 6.5 ml, for instance less than 6 ml, for instance less than 5.5 ml, for instance less than 5 ml, for instance less than 4.5 ml, for instance less than 4 ml, for instance less than 3.5 ml, for instance less than 3 ml, for instance less than

2. 5 ml, for instance less than 2 ml, for instance less than 1.5 ml, for instance less than 1 ml, for instance less than 0.5 ml, for instance less than 0.25 ml.

26. The sensor system of any of the preceding claims wherein the system is an ambulatory system.

27. The sensor system of any of the preceding claims wherein the system comprises the means to calculate the creatinine level/creatinine clearance rate/glomerular filtration rate.

28. The sensor system according to any of the preceding claims further comprising means to deliver an agent, optionally a contrast agent or a drug or creatinine, or creatine, or sarcosine, optionally wherein the means is a drug pump,
optionally wherein the drug is selected from the group consisting of immunosuppressants; chemotherapy agents such as platinum agents; antimicrobials such as the glycopeptides vancomycin and teicoplanin, and penicillin; and opioid analgesics such as morphine, diamorphine and codeine;
optionally wherein the amount of agent delivered is adjusted based on the calculated creatinine level/creatinine clearance rate/glomerular filtration rate.

29. The sensor system according to any of the preceding claims wherein the system further comprises a second sensor and optionally a second means to obtain a second sample, wherein the second sample is contacted with a second sensing reagent that comprises creatinase and sarcosine oxidase prior to detection at the second sensor, optionally wherein the system is arranged such that the second sensing reagent is added the to the second sample added more than 1, 2, 5, 10, 20, 30, 40, 50, 60, 70, 80, 90, 100, 110, 120, 130, 140, 150, 160, 170, 180, 190, 200, 225, 250 seconds, 5, 5.5, 6, 6.5, 7.5, 8, 8.5, 9, 9.5 or 10 minutes prior to contact with the sensor.

30. The sensor system according to claim 29 wherein the system comprises means to subtract the data obtained from the second sensor from the data obtained from the first sensor.

31. The sensor system according to any of the preceding claims wherein the first sensor captures data continuously.

32. The sensor system according to any of the preceding claims wherein the first sensor captures data at least every 24 hours, or at least every 22 hours, for example at least every 20 hours, for example at least every 18 hours, for example at least every 16 hours, for example at least every 14 hours, for example at least every 12 hours, for example at least every 10 hours, for example at least every 8 hours, for example at least every 6 hours, for example at least every 5 hours, for example at least every 4 hours, for example at least every 3 hours, for example at least every 2 hours for example at least every 1.5 hours, for example at least every 1 hour, for example at least every 50 minutes, for example at least every 45 minutes, for example at least every 40 minutes, for example at least every 35 minutes, for example at least every 30 minutes, for example at least very 25 minutes, for example at least every 20 minutes, for example at least every 15 minutes, for example at least every 10 minutes, for example at least every 5 minutes, for example at least every 2 minutes, for example at least every 1.5 minutes, for example at least every 60 seconds, for example at least every 45 seconds, for example at least every 30 seconds, for example at least every 15 seconds, for example at least every 10 seconds, for example at least every 5 seconds, for example at least every 2 seconds, for example at least every 1 second for example at least every 0.5 seconds.

33. A composition comprising any two of or all of the enzymes sarcosine oxidase, creatininase and creatinase.

34. The composition according to claim 33 comprising all of sarcosine oxidase, creatininase and creatinase.

35. The composition of claim 33 or 34 wherein at least one, optionally two, optionally all of the enzymes are not immobilised, optionally wherein all of the enzymes are in solution.

36. The composition according to claim 35 wherein the sarcosine oxidase, creatininase and creatinase are in solution.

37. The composition of claim 33-36 wherein the composition comprises a buffer.

38. The composition of claim 37 wherein the buffer is not a phosphate buffer or PBS, and/or is not a Tris buffer, and/or is not tetraborate and/or is not HEPES.

39. The composition of any one of claim 37 or 38 wherein the buffer is selected from the group consisting of EPPS, HEPBS, POPSO, HEPPSO and MOBS.

40. The composition of any one of claims 37-39 wherein the buffer has a pKa of between 7.0-9.0, optionally between 7.3-8.95, optionally 8.5.

41. The composition according to any one of claims 33-40 wherein the composition or the buffer is at a pH of between 7.0-9.0, optionally between 7.3-8.95, optionally 8.5.

42. The composition according to any one of claims 33-41 wherein the composition comprises EPPS at pH 8.0-8.5, optionally 50 mM EPPS at pH 8.0-8.5, optionally 50 mM EPPS at pH 8.0 or 50 mM EPPS at pH 8.5.

43. The composition of any one of claims 33-42 further comprising urease and/or uricase and/or means to detect Cystatin C and/or means to detect albumin.

44. The composition of any of the preceding claims wherein the creatininase is from Sorachim catalogue number CNH-311; and/or the creatinase is from Sorachim catalogue number CRH-211; and/or the sarcosine oxidase is from Sorachim catalogue number SAO-351.

45. The composition of any of the preceding claims wherein the concentration of creatininase and/or creatinase and/or sarcosine oxidase is such that in the final reaction mix the concentration of creatininase is at least 300 U/ml, and/or the concentration of creatinase is at least 120 U/ml and the concentration of sarcosine oxidase is at least 10 U/ml.

46. The composition of any of the preceding claims wherein the composition is such that the final mixed solution that results from the mixing of a sample which contains creatinine and the composition of any of the preceding claims comprises creatininase, creatinase, and sarcosine oxidase at a ratio of between 10:5:1 and 49:8:1 U/ml.

47. The composition of any of the preceding claims wherein the composition is such that the final mixed solution that results from the mixing of a sample which contains creatinine and the composition of any of the preceding claims comprises creatininase, creatinase, and sarcosine oxidase in the amounts of 600 U/ml, 300 U/ml and 60 U/ml, optionally wherein the composition is at pH 8.5.

48. A method for the determination of the level of creatinine in a sample from a human or animal subject, wherein the method comprises the use of the composition or sensor system according to any of the preceding claims, optionally wherein the sample is a dialysate or a microdialysate.

49. A method for the determination of the creatinine level and/or the creatinine clearance rate and/or the glomerular filtration rate wherein the method comprises the use of the composition or sensor system according to any of the preceding claims, optionally wherein the sample is a dialysate or a microdialysate.

50. A method for the real-time determination of the level of the creatinine level and/or the creatinine clearance rate and/or the glomerular filtration rate in a sample from a human or animal subject, wherein the method comprises the use of the composition of sensor system according to any of the preceding claims, optionally wherein the sample is a dialysate or a microdialysate.

51. A method for diagnosing a subject as having acute or chronic kidney disease, the method comprising determining the creatinine level and/or the creatinine clearance rate and/or the glomerular filtration rate according to any of the preceding methods, optionally further comprising treating the subject for acute or chronic kidney disease or stopping treatment with a drug that is contraindicated or dangerous in acute or chronic kidney disease, optionally wherein the drug is selected from the group consisting of
immunosuppressants; chemotherapy agents such as platinum agents; antimicrobials such as the glycopeptides vancomycin and teicoplanin, and penicillin; and opioid analgesics such as morphine, diamorphine and codeine.

52. The method of any of the preceding claims wherein determination of the level of the creatinine level and/or the creatinine clearance rate and/or the glomerular filtration rate is determined following administration of an amount of creatinine and/or creatine and/or sarcosine, optionally prior to and following administration of a drug.

53. The method of any of the preceding claims wherein the method further comprises administration of a dosage of a drug, wherein the dosage has been determined based on the creatinine level and/or the creatinine clearance rate and/or the glomerular filtration rate determined by the sensor system.

54. A method for monitoring a kidney for transplant, said method comprising perfusing the kidney and administering an amount of creatinine and/or creatine and/or sarcosine into the system, and determining the creatinine clearance rate using the composition and/or system and/or methods of any of the preceding claims.

55. A method for monitoring kidney function in a recipient of a transplant wherein the creatinine level and/or the creatinine clearance rate and/or the glomerular filtration rate is determined by use of the composition, sensor system and/or methods of any of the preceding claims.

56. A kit comprising:
any two or all of creatininase, creatinase and sarcosine oxidase; and/or
a composition according to any of the preceding claims;
creatinine and/or creatine and/or sarcosine; and/or
at least one waste receptacle;
a buffer, optionally a buffer according to any of the preceding claims;
a microdialysis probe; and/or
at least one, optionally at least two precision pumps.
